(19) United States
(12) Patent Application Publication
(10) Pub. No.: US 2009/0169540 A1
(43) Pub. Date: Jul. 2, 2009

(54) Use Of An Antagonist Of Epac For Treating Human Cardiac Hypertrophy

(75) Inventors: Frank Lezoualc'h, Sevres (FR); Eric Morel, Montfort L' Amaury (FR); Monique Gastineau, Wissous (FR); Gregoire Vandecasteele, Paris (FR)

Correspondence Address: YOUNG & THOMPSON, 209 Madison Street, Suite 500, ALEXANDRIA, VA, 22314, US

(73) Assignee: INSERM (Institute National De La Sane Et De La Recherche Medicle), Paris Cedex 13 (FR)

(21) Appl. No.: 11/885,452

(22) PCT Filed: Mar. 2, 2006
(86) PCT No.: PCT/EP2006/001903

§ 371 Date: Jul. 22, 2008

Related U.S. Application Data
(60) Provisional application No. 60/657,707, filed on Mar. 3, 2005.

(51) Int. Cl. A61K 39/395; C07K 16/18; C07H 19/067; C07D 313/00; A61K 31/7105; G01N 33/68; C12Q 1/02; A01K 67/027

(52) U.S. Cl. 424/130.1; 530/387.1; 536/26.7; 549/270; 514/44; 436/86; 435/29; 800/9

(57) ABSTRACT

The present invention relates to the use of at least one Epac (Exchange Protein directly Activated by cAMP) antagonist for the manufacture of a medicament intended for the prevention or the treatment of pathologies selected from the list comprising cardiac hypertrophy, cardiac arrhythmias, valvulopathies, diastolic dysfunction, chronic heart failure, ischemic heart failure, and myocarditis.

### FIELD OF THE INVENTION

The present invention relates to methods and compositions for treating Human Cardiac Hypertrophy (HCM). More specifically, the present invention relates to the use of an antagonist of Epac for treating HCM.

### BACKGROUND

Physiologic hypertrophy, such as an exercise-induced cardiac hypertrophy, represents a favorable adaptive change in the heart that accommodates to the increases in body demand, and does not lead to heart failure. In contrast, pathologic hypertrophy, such as pressure overload-induced hypertrophy, is a maladaptive response to pathologic stimuli that, if not ameliorated, usually leads to heart failure.

Human cardiac hypertrophy (HCM) affects an estimated 600,000 to 1.5 million Americans, or one in 500 people. It is more prevalent than multiple sclerosis, which affects one in 700 people. HCM is the most common cause of sudden cardiac death in people under age 30.

Human cardiac hypertrophy (HCM) often develops as a by-product of hypertension or valvular heart disease. Adult cardiomyocytes are unable to divide and respond to stress and growth stimuli by increasing their rate of protein synthesis, resulting in increased cell volume (Sadoshima et al. 1997). Growth of individual cardiomyocytes results in thickening of the heart. Cardiac hypertrophy is a potent risk factor for the development of cardiac arrhythmias, diastolic dysfunction, congestive heart failure, and death. (Hennersdorf et al. 2001; Vakili et al. 2001).

To date a variety of drug treatments are currently used for treating human cardiac hypertrophy.

For instance beta-blocking drugs slow the heart beat and reduce its force of contraction. These drugs usually relieve chest pain, breathlessness and palpitation, but occasionally excessive heart rate slowing with these drugs can cause fatigue.

The second major group of drugs used are the calcium antagonists or calcium channel blockers. Within this group verapamil is the drug which has been most commonly used in HCM. It improves the filling of the heart and like beta-blockers, reduces symptoms such as chest pain, breathlessness and palpitations. Also, like beta-blockers, verapamil can cause excessive slowing of the heart rate and lower blood pressure.

Anti-arrhythmic drugs have been also used for treating of cardiac hypertrophy. These drugs might be used when an arrhythmia such as tachycardia is detected and felt to be important in an individual case. Of these anti-arrhythmic drugs, Amiodarone (Cordarone) is the most commonly used in hypertrophic cardiomyopathy. It is a powerful and effective drug. But it does have several potentially serious side effects, including pulmonary toxicity (2%-7% in some studies, but as high as 10%-17% in some reports) liver function test abnormalities (4%-9%), hyperthyroidism (about 2%), and hypothyroidism (2%-4% in some cases, but as high as 8%-19% in some series), proarrhythmia (2%-5%) and optic neuropathy, which can lead to blindness.

Thus, there is a permanent need in the art to find alternative drugs for treating HCM that preferably do not show the side effects as described above.

It is now well admitted that Ca2+-sensitive signaling pathways play crucial roles in cardiomyocyte hypertrophy (Frey et al, 2000). Two prominent Ca2+-dependent pathways involve the Ser/Thr protein phosphatase calcineurin (Liang et al, 2003; Molkentin, 2004) and Ca2+/calmodulin-dependent protein kinase II (CaMKII) (Zhang & Brown, 2004). Activation of calcineurin by Ca2+ results in the dephosphorylation and nuclear translocation of cytoplasmic nuclear factor of activated T cells (NFAT) transcription factors which then upregulate transcription of hypertrophic genes. Calcineurin can also relieve the inhibition of class II histone deacetylases (HDACs) on the myocyte enhancer factor 2 (MEF2), thereby allowing this transcription factor to induce hypertrophic gene expression. In addition, CaMKII is known to activate MEF2 upon HDACs phosphorylation (McKinsey & Olson, 2004). To date, the participation of small G proteins of the Rho family in the regulation of these hypertrophic signaling cascades is not well defined.

Among the superfamily of small G proteins, the Rho-family which includes Rho, Rac and Cdc42 has attracted much interest for they have been shown to play key roles in the generation of cytoskeletal structures (Hall, 1998). Indeed, Rho is important for the formation of stress fibers and focal adhesions in fibroblasts, whereas Rac and Cdc42 are involved in the regulation of more dynamic structures such as membrane ruffles, lamellipodia and filopodia (Hall, 1998). Several studies have pointed out the role of Rho proteins in the development of cardiomyocyte hypertrophy (Clerk & Sugden, 2000). For instance, two potent hypertrophic stimuli, endothelin 1 (ET-1) and phenylephrine (PE) induce rapid activation of endogenous Rac in neonatal cardiomyocytes (Clerk et al, 2001). In addition, adenoviral infection of cardiomyocytes with a constitutive active form of Rac (RacG12V) also increases ANF expression and protein synthesis, and promotes morphological changes associated with myocyte hypertrophy (Pracyk et al, 1998). In vivo evidence for the role of Rho proteins in cardiac hypertrophy came from transgenic mice specifically expressing RacG12V in the heart. These mice develop a dilated cardiomyopathy associated with deregulation of cardiomyocyte focal adhesions (Sussman et al, 2000). These data suggest that Rho proteins, especially Rac control hypertrophic response and are likely to be involved in cardiac remodelling, and the pathogenesis of cardiomyopathy characterized by cellular enlargement.

Cyclic adenosine 3′,5′-monophosphate (cAMP) is one of the most important second messenger in the heart because it regulates many physiological processes such as cardiac contractility, relaxation and automaticity. Classically, these effects are attributed to activation of hyperpolarization-activated cyclic nucleotide-gated (HCN) channels and protein kinase A (PKA) by cAMP (Fimia & Sassone-Corsi, 2001). PKA is considered to be the essential effector molecule mediating the many physiological effects of Gs-coupled-receptors. A classical example is the stimulation of cardiac β-adrenergic receptors in which PKA phosphorylates several key proteins involved in excitation-contraction coupling, such as L-type Ca²⁺ channels, phospholamban, ryanodine receptors (RyR) and troponin I (Bers & Ziolo, 2001).

The recent discovery of Epac (exchange proteins directly activated by cAMP) as proteins which are directly activated by cAMP has broken the dogma surrounding cAMP and PKA (U.S. Pat. No. 6,987,004; Bos, 2003; de Rooij et al, 1998; Kawasaki et al, 1998). Epac proteins are guanine nucleotide exchange factors (GEFs) that bind cAMP with affinities similar to that of the regulatory subunit of PKA (de Rooij et al, 1998; Kawasaki et al, 1998). They have been shown to function as GEFs for the Ras-like small GTPases Rap1 and Rap2 and are directly activated by cAMP in a PKA independent manner (Bos, 2003). There are two isoforms of Epac, Epac 1 and Epac 2 both consisting of a regulatory and a catalytic region (de Rooij et al, 1998; Kawasaki et al, 1998). Epac1 is highly expressed in the heart (Kawasaki et al, 1998). Epac 2 has an additional cAMP binding domain which is dispensable for cAMP-induced Rap activation (de Rooij et al, 2000). Following cAMP binding, Epac catalyses the exchange of GDP for a GTP of the small GTPases Rap, allowing interaction with their target effectors (Rehmann et al, 2003). Recent studies indicate that Epac is involved in cell adhesion (Enserink et al, 2004; Rangarajan et al, 2003), neurite extension (Christensen et al, 2003), and regulates the amyloid precursor protein and insulin secretion (Maillet et al, 2003; Ozaki et al, 2000). A recent study has provided experimental evidence that Epac1 stimulates the activity of the small GTPase, Rac in a cAMP-dependent but PKA-independent manner in neuronal cells (Maillet et al, 2003).

### SUMMARY

The Inventors now surprisingly show that Epac1 stimulates the activity of the small GTPase, Rac and increases the expression of hypertrophic gene markers in cultured cardiomyocytes. Furthermore, the Inventors demonstrate that Epac1 induces cardiomyocyte hypertrophy. This process is associated with intracellular [Ca2+] mobilization and the activation of the transcription factors NFAT and MEF2. Altogether, these findings identify the cAMP-binding protein, Epac as a new positive regulator of cardiac growth.

A first aspect of this invention thus resides in the use of an antagonist of Epac for the manufacture of a medicament for treating cardiac hypertrophy, and especially, human cardiac hypertrophy.

A further object of this invention resides in a method for treating cardiac hypertrophy, especially human cardiac hypertrophy, comprising administering at least one antagonist of Epac to the subject.

A further object of the invention resides in a pharmaceutical composition useful for treating cardiac hypertrophy, and especially human cardiac hypertrophy, comprising at least one antagonist of Epac.

In one embodiment, the antagonist of Epac is a compound natural or not that impedes or limits the activation of Epac by cAMP or hypertrophic stimuli such as angiotensin II, endothelin I and phenylephrine. In a particular embodiment, said antagonist is a cAMP analog, that competes with cAMP for binding to Epac, but to thereupon either block or significantly inhibit the biological response induced by cAMP or hypertrophic stimuli such as angiotensin II, endothelin I and phenylephrine. cAMP analog candidates are those compounds that have an affinity for binding to Epac that is either not significantly different from, or higher than cAMP, and that induce no, or a significantly lower level of biological response than native cAMP.

In a further embodiment, the antagonist of Epac, is a compound that is able to inhibit the expression of Epac. In a particular embodiment, said antagonist is an antisense oligonucleotide, a siRNA or a ribozyme.

A further object of the invention resides in a method for screening a compound, and especially a cAMP analog, that affects the activation of Epac.

In one embodiment, the screening method involves the steps of:
a) incubating Epac with i) at least one effector of Epac, chosen among, but not limited to, Rap1, Rap2, Rac or Ras, ii) cAMP and iii) said compound to be tested,
b) assessing the activation of said effector by Epac,
   wherein a lower activation, in comparison with the activation provided in absence of said compound to be tested, indicates that said compound is an antagonist of Epac. The assay of effector activation could be performed as hereunder described in Example 1 “Rac activation assay”, ie wherein said effector is coupled in frame with GST. In a further embodiment said effector is Rap1 that can be loaded with fluorescently labelled 2′,3′-bis(O)—N-methylantharanoloyl-guanosinediphosphate (mGDP). In this particular embodiment, the activation of Rap1 is assessed by detecting in real time the release of mGDP with a fluorescence spectrometer as previously described (Van den Berghe et al., 1997).

In a further embodiment, the screening method involves the step of:

a) providing cardiomyocytes that either constitutively expressed an activated form of Epac or Epac wild type, or have been treated with hypertrophic stimuli such as angiotensin II, endothelin I and phenylephrine.
b) exposing said cardiomyocyte to one of the compounds to be tested
c) assessing the effects of said compound on the hypertrophy properties of said cardiomyocytes
wherein the inhibition of the hypertrophic properties indicates that said compound is an antagonist of Epac.

In a particular embodiment, cardiomyocyte hypertrophy properties are determined by measuring cell size, protein content and/or the expression of mRNA coding for hypertrophic gene markers such as the natriuretic factor or NFAT transcriptional activity.

In a further embodiment, the screening method can result in the combination of the two above described methods.

A further object of the invention resides in a non human model of cardiac hypertrophy wherein cardiac hypertrophy has been induced by the specific expression of a positive dominant form of Epac, and especially Epac1. In a particular embodiment, said model is a mouse. In another particular embodiment, said positive dominant form of Epac is under the control of a cardiac specific promoter such as α-Myosin Heavy Chain promoter. In a further embodiment the positive dominant form of Epac1 result in Epac-ΔcAMP which contains a deletion of the first 322 amino acids of Epac1 (de Rooij et al., 1998).

In one embodiment, Epac is the protein shown in SEQ ID NO: 2 (Epac1, Accession: NM_{—}006105) or SEQ ID NO: 4 (Epac2, Accession: NM_{—}007023), or a partial protein thereof, or an ester, amide or salt thereof.

### DETAILED DESCRIPTION

The present invention relates to the use of at least one Epac (Exchange Protein directly Activated by cAMP) antagonist for the manufacture of a medicament intended for the prevention or the treatment of pathologies selected from the list comprising cardiac hypertrophy, cardiac arrhythmias, valvulopathies, diastolic dysfunction, chronic heart failure, ischemic heart failure, and myocarditis.

An antagonist of Epac is herein defined as a compound that is able to inhibit the expression, the activation or the activity of Epac.

An antagonist of Epac that inhibits the expression of Epac means that said antagonist impedes the process of transcription of the Epac gene and/or of the translation of the mRNA of Epac into the Epac protein.

An antagonist of Epac that inhibits the activation of Epac means that said antagonist impedes the activators of Epac to activate Epac.

An antagonist of Epac that inhibits the activity of Epac means that said antagonist impedes Epac function. Such an antagonist may block the catalysis by Epac of the exchange of GDP for a GTP.

The invention relates in particular to the use as defined above, wherein the antagonist is selected from the list comprising Epac activation inhibitors, Epac activity inhibitors, Epac intracellular localization disruption agents, and Epac expression inhibitors.

An “Epac expression inhibitor” impedes or decreases the expression of the protein Epac. Said inhibitor may block or decrease the transcription and/or the translation process of Epac. The inhibition of Epac expression can be assessed by Western blot or reverse transcriptase polymerase chain reaction.

An “Epac activation inhibitor” impedes the activation of Epac, particularly by the activators of Epac such as cAMP. The inhibition of Epac activation can be assessed by measuring the GTP form of its effector, Rap1 or Rap2, using pull down assay as described in Maillet et al., 2003.

An “Epac activity inhibitor” is a compound which impedes the normal function of Epac, such as the exchange of GDP for a GTP or the activation of the c-Jun NH2-terminal kinase.

The inhibition of Epac activity can be assessed by measuring either the GTP form of its effector, Rap1 or Rap2 using pull down assay or the activation of c-Jun NH2-terminal kinase using a kinase assay as described in Hochbaum et al. (2003).

An “Epac intracellular localization disruption agent” is a compound which impedes the proper cellular localization of Epac. Epac cellular distribution can be assessed by immunocytochemistry using Epac selective antibodies.

The invention further relates to the use as defined above, wherein the antagonist is an Epac activation inhibitor selected from the list comprising:
antibodies, fragments thereof, or aptamers, directed against Epac cAMP binding sites,
cAMP analogues, such as cAMP derivatives or 8-(4-chlorophenylthio)-2′-O-methyladenosine-3′-5-cyclic monophosphate (8-CPT-2′-O-Me-cAMP) derivatives,
Brefeldin A or derivatives thereof.

Compounds capable of inhibiting the activation of Epac include in particular those able to interact with natural agonists of Epac, such as cAMP, and/or to interact in the binding of said agonists to Epac, and/or to inhibit the activation of Epac resulting from said binding.

The term “agonist of Epac” refers to compounds that bind to Epac and activate Epac through this binding.

An inhibitor of activation of Epac may be an antibody or fragment thereof, or an aptamer directed against the Epac cAMP binding sites, thus impeding the binding of said cAMP to Epac.

In the present invention, the term “antibody” refers to a polyclonal or monoclonal antibody. The terms “polyclonal” and “monoclonal” refer to the degree of homogeneity of an antibody preparation, and are not intended to be limited to a particular method of production.

A mammal, such as a rabbit, a mouse, or a hamster, can be immunized with an immunogenic form of the protein, such as the entire protein or a part of it. The protein or part of it can be administered in the presence of an adjuvant.

The term “immunogenic” refers to the ability of a molecule to elicit an antibody response. Techniques for conferring immunogenicity to a protein or part of it which is not itself immunogenic include conjugation to carriers or other techniques well known in the art.

The immunization process can be monitored by detection of antibody titers in plasma or serum. Standard immunoassays, such as ELISA can be used with the immunogenic protein or peptide as antigen to assess the levels of antibody.

According to the invention, an antibody which is an activation inhibitor of Epac is directed against Epac cAMP binding sites.

In the present invention, the term “aptamer” refers to a nucleic acid molecule or oligonucleotide sequence that binds specifically to the Epac protein. Aptamers may be DNA or RNA and may include nucleotide derivatives. Aptamers may be a single strand or a double strand of usually 16 to 60 nucleotides long. The method called SELEX (Systematic Evolution of Ligands by Exponential Enrichment) is generally used to select and identify aptamers exhibiting affinity for the target.

According to the invention, an aptamer which is an activation inhibitor of Epac binds specifically to Epac cAMP binding sites.

As used herein, the term “oligonucleotide” refers to a nucleic acid, generally of at least 10, preferably at least 12, more preferably at least 15, and still preferably at least 20 nucleotides, preferably no more than 100 nucleotides, still preferably no more than 70 nucleotides, and which is hybridizable to a Epac genomic DNA, cDNA, or mRNA.

An inhibitor of activation of Epac can consist in an analog of cAMP which impedes the binding of said cAMP to Epac. In a particular embodiment, said antagonist is a cAMP analog, that competes with cAMP for binding to Epac, but to thereupon either block or significantly inhibit the biological response induced by cAMP or hypertrophic stimuli such as angiotensin II, endothelin I and phenylephrine. cAMP analog candidates are those compounds that have an affinity for binding to Epac that is either not significantly different from, or higher than cAMP, and that induce no, or a significantly lower level of biological response than native cAMP.

Said analog of cAMP can be identified by the screening methods described hereinafter.

Another inhibitor of Epac activation may be Brefeldin A, a small hydrophobic compound produced by toxic fungi. Brefeldin A is a macrocyclic lactone exhibiting a large range of antibiotic activity. Brefeldin A can bind at the Rap-GDP/Epac interface, thus freezing the complex in an abortive conformation that cannot proceed to nucleotide dissociation.

The invention also relates to the use as defined above, wherein the antagonist is an Epac activity inhibitor, in particular an inhibitor of the Guanine nucleotide Exchange Factor (GEF) domain of Epac, or an inhibitor of the Ras Exchange Motif (REM) domain of Epac, selected from the list comprising:
antibodies, fragments thereof, or aptamers, directed against the GEF domain or the REM domain of Epac,
Brefeldin A or derivatives thereof.

The Guanine nucleotide Exchange Factor domain (GEF) is located into the catalytic region of Epac protein and is involved in Epac catalytic activity. The GEF domain of Epac is located between amino acids 616-848 of Epac1 and 768-1005 of Epac2.

The Ras Exchange Motif (REM) domain is located into the catalytic region of Epac protein and participates to Epac catalytic activity. The REM domain of Epac is located between amino acids 342-466 of Epac1 and 495-615 of Epac2.

The invention also relates to the use as defined above, wherein the antagonist is an Epac intracellular localization disruption agent, selected from the list comprising:
antibodies, fragments thereof, or aptamers, directed against an Epac cellular localization domain, such as the Dishevelled Egl-10 Pleckstrin (DEP) domain,
Brefeldin A or derivatives thereof.

The term “Epac cellular localization domain” refers to a domain which is involved in membrane localization. An Epac cellular localization domain is for example the Dishevelled Egl-10 Pleckstrin (DEP) domain.

The invention also relates to the use as defined above, wherein the antagonist is an Epac expression inhibitor selected from the list comprising:
an antisense nucleic acid directed against Epac mRNAs,
a single stranded DNA, directed against Epac double strand DNA,
a double stranded RNA, a siRNA or a shRNA, comprising Epac nucleic acid sequences,
a ribozyme directed against Epac in mRNAs.

Inhibitors of the expression of Epac include for instance antisense oligonucleotides, or interfering RNAsi, or ribozymes, targeting the Epac gene.

The term “gene” means a DNA sequence that codes for or corresponds to a particular sequence of amino acids which comprise all or part of one or more proteins or enzymes, and may or may not include regulatory DNA sequences, such as promoter sequences, which determine for example the conditions under which the gene is expressed. A “promoter” or “promoter-sequence” is a DNA regulatory region capable of binding RNA polymerase in a cell and initiating transcription of a downstream (3′ direction) coding sequence. Some genes, which are not structural genes, may be transcribed from DNA to RNA, but are not translated into an amino acid sequence. Other genes may function as regulators of structural genes or as regulators of DNA transcription. In particular, the term gene may be intended for the genomic sequence encoding a protein, i.e. a sequence comprising regulator, promoter, intron and exon sequences.

A “coding sequence” or a sequence “encoding” an expression product, such as a RNA, polypeptide, protein, or enzyme, is a nucleotide sequence that, when expressed, results in the production of that RNA, polypeptide, protein, or enzyme, i.e., the nucleotide sequence encodes an amino acid sequence for that polypeptide, protein or enzyme. A coding sequence for a protein may include a start codon (usually ATG) and a stop codon.

As used herein, the term “Epac gene” denotes the gene encoding for Epac (exchange proteins directly activated by cAMP) of any species, especially human Epac, but also other mammals or vertebrates to which the invention can apply. Unless otherwise indicated, the term “Epac” is used indifferently to designate the Epac gene or the encoded protein Epac throughout the text. There are two isoforms of Epac, Epac 1 (also named Rap guanine nucleotide exchange factor (GEF) 3) and Epac 2 (also named Rap guanine nucleotide exchange factor (GEF) 4) such as described in de Rooij et al, 1998 and Kawasaki et al, 1998. In addition, a related protein named Repac (for related to Epac) has been identified by Ichiba et al. (1999). Repac shows close sequence similarity to Epac 2 and lacks the regulatory sequences present in Epac1 and Epac2 (Ichiba et al., 1999). Homo sapiens Epac 1 gene is localized on chromosome 12. The nucleotide and amino acids sequences of Epac1, SEQ ID NO: 1 and 2 respectively, are deposited in Genebank under accession number NM_{—}006105. The nucleotide and amino acids sequences of Epac 2, SEQ ID NO: 3 and 4 respectively, are deposited in Genebank under accession number NM_{—}007023. Epac 2 is mapped to human chromosome 2q31. The nucleotide and amino acids sequences of Repac, SEQ ID NO: 5 and 6 respectively, are deposited in Genebank under accession number BC039203. Repac is mapped to human chromosome 7p15.3.

Antisense molecules can be modified or unmodified RNA, DNA, or mixed polymer oligonucleotides and primarily function by specifically binding to matching sequences resulting in inhibition of peptide synthesis. The antisense oligonucleotide binds to target RNA by Watson Crick base-pairing and blocks gene expression by preventing ribosomal translation of the bound sequences either by steric blocking or by activating RNase H enzyme. Antisense molecules can also alter protein synthesis by interfering with RNA processing or transport from the nucleus into the cytoplasm. In addition, antisense deoxyoligoribonucleotides can be used to target RNA by means of DNA-RNA interactions, thereby activating RNase H, which digests the target RNA in the duplex. Antisense DNA can be expressed via the use of a single stranded DNA intracellular expression vector or equivalents and variations thereof.

Antisense nucleic acids are useful for regulating and controlling the expression of the Epac protein gene in vivo and in vitro, and are also useful for the treatment of the diseases disclosed above. Technologies related to such antisense RNAs and gene therapies are known to the skilled man. The novel antisense oligonucleotides complementary to any sequence of the human Epac RNA, which according to the broadest definition can be of a length ranging from 7 to 40 nucleotides, have preferably a length ranging from 15 to 25 nucleotides, most preferably about 20 nucleotides.

The term “complementary” means that the antisense oligonucleotide sequence can form hydrogen bonds with the target mRNA sequence by Watson-Crick or other base-pair interactions. The term shall be understood to cover also sequences which are not 100% complementary. It is believed that lower complementary, even as low as 50% or more, may work. However, 100% complementary is preferred.

Single stranded DNA can be designed to bind to genomic DNA in a sequence specific manner. Triplex Forming Oligonucleotides (TFO) are comprised of pyrimidine-rich oligonucleotides which bind DNA helices through Hoogsteen Base-pairing. The resulting triple helix composed of the DNA sense, DNA antisense, and TFO disrupts RNA synthesis by RNA polymerase.

Double-stranded RNAs can suppress expression of homologous genes through an evolutionarily conserved process named RNA interference (RNAi). One mechanism underlying silencing is the degradation of target mRNAs by an RNP (RiboNucleoProtein) complex, which contains short interfering RNAs (siRNAs) as guides to substrate selection.

The term “siRNA” refers to a short (typically less than 30 nucleotides long) double stranded RNA molecule. Typically, the siRNA modulates the expression of a gene to which the siRNA is targeted. Selection of a suitable small interfering RNA (siRNA) molecule requires knowledge of the nucleotide sequence of the target mRNA, or gene from which the mRNA is transcribed. The siRNA molecules of the invention are typically between 10-30 nucleotides in length, and preferably between 18-23 nucleotides in length. The siRNA molecules may comprise a sequence identical or at least 90% identical to any portion of the target gene whose expression is to be modulated including coding and non-coding sequences.

To provide a siRNA that can specifically suppress the expression of Epac, the following guidelines are used according to Elbashir et al., 2002: 1) Selection of the target region from the open reading frame (ORF) of the cDNA sequence preferably 50 to 100 nucleotides downstream of the start codon. 2) Determination of a 21 nucleotide sequence in the target mRNA that begins with an AA dinucleotide (Elbashir et al., 2001). Thus sequences are 5′-AA(N19)UU, where N is any nucleotide. Sequences must contain approximately 50% G/C. 3) Blast-search (www.ncbi.nih.go/BLAST) the selected siRNA sequences against EST libraries or mRNA sequences of the respective organism to ensure that only a single gene is targeted. Any target sequences with more than 16 to 17 contiguous base pairs of homology to other coding sequences must be eliminated. 4) Synthesis of several siRNA sequences are advisable to control for the specificity of the knock-down experiments.

A short hairpin RNA is a simple strain RNA, characterized in that the two ends of said RNA are complementary and can hybridize together, thus forming an artificial double strand RNA with a loop between the two ends.

Ribozymes are RNA molecules possessing the ability to specifically cleave other single-stranded RNA. Through the modification of nucleotide sequences which encode these RNAs, it is possible to engineer molecules that recognize specific nucleotide sequences in an RNA molecule and cleave it. A major advantage of this approach is that, because the ribozymes are engineered to be sequence-specific, only mRNAs with sequences complementary to the construct containing the ribozyme are inactivated. There are two basic types of ribozymes namely, tetrahymena-type and “hammerhead”-type. Tetrahymena-type ribozymes recognize sequences which are four bases in length, while “hammerhead”-type ribozymes recognize base sequences from about 3 to 18 bases in length. The longer the recognition sequence, the greater the likelihood that the sequence will occur exclusively in the target mRNA species. Consequently, hammerhead-type ribozymes are preferable to tetrahymena-type ribozymes for inactivating a specific mRNA species.

In accordance with the present invention, ribozyme, siRNAs, single strand DNA, or antisense oligonucleotides may be produced by expression of DNA sequences cloned into plasmid or retroviral vectors. Using standard methodology known to those skilled in the art, it is possible to maintain the ribozyme, siRNA or antisense oligonucleotides in any convenient cloning vector.

Various genetic regulatory control elements may be incorporated into ribozyme, siRNA, single stranded. DNA, or antisense oligonucleotides expression vectors to facilitate propagation in cardiac cells. For instance, the cardiac specific expression of the siRNA may be driven by a cardiac specific promoter such as the α-Myosin Heavy Chain promoter.

A “vector” is a replicon, such as a plasmid, cosmid, bacmid, phage or virus, to which another genetic sequence or element (either DNA or RNA) may be attached so as to bring about the replication of the attached sequence or element.

A “replicon” is any genetic element, for example, a plasmid, cosmid, bacmid, phage or virus, that is capable of replication largely under its own control. A replicon may be either RNA or DNA and may be single or double stranded.

An “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

According to another embodiment, the present invention relates to a method for treating a pathology selected from the list comprising cardiac hypertrophy, cardiac arrhythmias, valvulopathies, diastolic dysfunction, chronic heart failure, ischemic heart failure, and myocarditis, in a patient, comprising administering to said patient a therapeutically effective amount of at least one Epac antagonist.

The invention relates to the method as defined above, wherein the antagonist is selected from the list comprising Epac activation inhibitors, Epac activity inhibitors, Epac intracellular localization disruption agents, and Epac expression inhibitors.

The invention also relates to a method as defined above, wherein the antagonist is an Epac activation inhibitor selected from the list comprising:
antibodies, fragments thereof, or aptamers, directed against Epac cAMP binding sites,
cAMP analogues, such as cAMP derivatives or 8-(4-chlorophenylthio)-2′-O-methyladenosine-3′-5-cyclic monophosphate (8-CPT-2′-O-Me-cAMP) derivatives,
Brefeldin A or derivatives thereof.

The daily dosage of said antagonists may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the patient to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 10 mg/kg of body weight per day. It will be understood, however, that the specific dose level and frequency of dosage for any particular patient may be varied and will depend upon a variety of factors including the activity of the specific compound employed, the metabolic stability, and length of action of that compound, the age, the body weight, general health, sex, diet, mode and time of administration, rate of excretion, drug combination, the severity of the particular condition, and the host undergoing therapy.

In the pharmaceutical compositions of the present invention for oral, sublingual, subcutaneous, intramuscular, intravenous, transdermal, local or rectal administration, the antagonist of Epac, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

In the pharmaceutical compositions of the present invention, the active principle is generally formulated as dosage units containing from 0.5 to 1000 mg, preferably from 1 to 500 mg, more preferably from 2 to 200 mg of said active principle per dosage unit for daily administrations.

The invention further relates to a method as defined above, wherein the antagonist is an Epac activity inhibitor, in particular an inhibitor of the Guanine nucleotide Exchange Factor (GEF) domain of Epac, or an inhibitor of the Ras Exchange Motif (REM) domain of Epac, selected from the list comprising:
antibodies, fragments thereof, or aptamers, directed against the GEF domain or the REM domain of Epac,
Brefeldin A or derivatives thereof.

The invention also relates to a method as defined above, wherein the antagonist is an Epac intracellular localization disruption agent, selected from the list comprising:
antibodies, fragments thereof, or aptamers, directed against an Epac cellular localization domain, such as the Dishevelled Egl-10 Pleckstrin (DEP) domain,
Brefeldin A or derivatives thereof.

The invention also relates to a method as defined above, wherein the antagonist is an Epac expression inhibitor selected from the list comprising:
an antisense nucleic acid directed against Epac mRNAs,
a double stranded RNA, a siRNA or a shRNA comprising Epac nucleic acid sequences,
a ribozyme directed against Epac mRNAs.

According to another embodiment, the present invention relates to a pharmaceutical composition comprising as active substance an Epac expression inhibitor selected from the list comprising:
an antisense nucleic acid directed against Epac mRNAs,
a double stranded RNA, a siRNA or a siRNA comprising Epac nucleic acid sequences,
a ribozyme directed against Epac mRNAs.
   in association with a pharmaceutically acceptable carrier.

Ribozyme, siRNA, or antisense oligonucleotides encoding vectors and constructs as described herein are generally administered to a subject as a pharmaceutical preparation. As used herein, the term “subject” denotes a mammal, such as a rodent, a feline, a canine, and a primate. Preferably a subject according to the invention is a human

The pharmaceutical preparation comprising the ribozyme, siRNA or antisense oligonucleotides molecules or vectors are conveniently formulated for administration with an acceptable medium such as water, buffered saline, ethanol, polyol (for example, glycerol, propylene glycol, liquid polyethylene glycol and the like), dimethyl sulfoxide (DMSO), oils, detergents, suspending agents or suitable mixtures thereof. The concentration of the ribozyme, siRNA or antisense oligonucleotides molecules or vectors in the chosen medium may depend on the hydrophobic or hydrophilic nature of the medium, as well as the length and other properties of the ribozyme siRNA or antisense oligonucleotides molecules or vectors. Solubility limits may be easily determined by one skilled in the art.

Ribozyme, siRNA or antisense oligonucleotides molecules and vectors encoding the same may be administered parenterally by intravenous injection into the blood stream, by subcutaneous, intramuscular, or intraperitoneal injection, or any other method known in the art. Pharmaceutical preparations for parenteral injection are commonly known in the art. If parenteral injection is selected as a method for administering the molecules or vectors, steps must be taken to ensure that sufficient amounts of the molecules or vectors reach their target cells to exert a biological effect. Several techniques have been used to increase the stability, cellular uptake and biodistribution of oligonucleotides. Ribozyme, siRNA or antisense oligonucleotides molecules of the present invention may be encapsulated in a lipophilic, targeted carrier, such as a liposome.

A pharmaceutical preparation of the invention may be formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art. Dosage units may be proportionately increased or decreased based on the weight of the patient. Appropriate concentrations for alleviation of a particular pathological condition may be determined by dosage concentration curve calculations, as known in the art.

In accordance with the present invention, the appropriate dosage unit for the administration of ribozyme-siRNA or antisense oligonucleotides molecules may be determined by evaluating the toxicity of the ribozyme, siRNA or antisense oligonucleotides molecules in animal models. Various concentrations of said molecules in pharmaceutical preparations may be administered to mice and the minimal and maximal dosages may be determined based on comparing obtaining desired results as opposed to side effects as a result of the treatment.

The pharmaceutical preparation comprising the molecules may be administered at appropriate intervals, for example, twice a day until the pathological symptoms are reduced or alleviated, after which the dosage may be reduced to a maintenance level.

The pharmaceutical composition is advantageously substantially pure. The term “substantially pure” refers to a preparation comprising at least 50-60% by weight of a given material (e.g. nucleic acid, oligonucleotide). More preferably, the preparation comprises at least 75% by weight, and most preferably 90-95% by weight of the given compound. Purity is measured by methods appropriate for the given compound (e.g. chromatographic methods, agarose or polyacrylamide gel electrophoresis, HPLC analysis, and the like).

The invention relates in particular to pharmaceutical compositions comprising as active substance an Epac activation inhibitor, an Epac activity inhibitor, or an Epac intracellular localization disruption agent, selected from the list comprising:
antibodies, fragments thereof, or aptamers, directed against Epac cAMP binding sites,
antibodies, fragments thereof, or aptamers, directed against the GEE domain or the REM domain of Epac,
antibodies, fragments thereof, or aptamers, directed against an Epac cellular localization domain, such as the DEP domain,
   in association with a pharmaceutically acceptable carrier.

The invention also relates to the use of Epac for screening compounds intended for the prevention or the treatment of pathologies selected from the list comprising cardiac hypertrophy, cardiac arrhythmias, valvulopathies, diastolic dysfunction, chronic heart failure, ischemic heart failure, and myocarditis.

These compounds are particularly chosen among Epac activation inhibitors, Epac activity inhibitors, Epac intracellular localization disruption agents, and Epac expression inhibitors.

The invention further relates to a method for screening compounds intended for the prevention or the treatment of pathologies selected from the list comprising cardiac hypertrophy, cardiac arrhythmias, valvulopathies, diastolic dysfunction, chronic heart failure, ischemic heart failure, and myocarditis, comprising the steps of:
contacting Epac with a compound to screen in the presence of cAMP or an Epac activating cAMP analogue,
assaying Epac activation,
selecting compounds which inhibit Epac activation, in particular compounds which inhibit at least 30% of Epac activation, as compared to Epac activation in the absence of said compounds.

The cAMP-dependent activation of Rap1 can be assessed to determinate the percentage of Epac activation.

The invention also relates to the method as defined above, wherein Epac is also contacted with an effector protein liable to be activated upon Epac activation, such as Rap1, Rap2, Rac or Ras, and Epac activation is assessed by measuring the activity of said effector protein.

The screening method thus involves the steps of
a) incubating Epac with i) at least one effector of Epac, chosen among, but not limited to, Rap1, Rap2, Rac or Ras, ii) cAMP and ii) said compound to be tested,
b) assessing the activation of said effector by Epac,
   wherein a lower activation, in comparison with the activation provided in absence of said compound to be tested, indicates that said compound is an antagonist of Epac. The assay of effector activation could be performed as hereunder described in Example 1 “Rac activation assay”, ie wherein said effector is coupled in frame with GST.

In a particular embodiment, the screening method comprises testing in vitro the effect of the compounds to inhibit Epac-induced cAMP-dependent Rap1 or Rap2 activation. Rap 1 is a well known effector of Epac. To do that, full-length Epac is incubated with Rap1 or Rap2 loaded with fluorescently labelled 2′,3′-bis(O)—N-methylantharanoloyl-guanosinediphosphate (mGDP) and the release of mGDP is detected in real time by a fluorescence spectrometer as previously described (Van den Berghe et al., 1997). The inhibition effect of the compounds on Epac-induced cAMP-dependent Ras or Rac activation can be assessed using GST pull down assay as previously described (Maillet et al., 2003).

According to another embodiment, the present invention relates to a method for screening compounds intended for the prevention or the treatment of pathologies selected from the list comprising cardiac hypertrophy, cardiac arrhythmias, valvulopathies, diastolic dysfunction, chronic heart failure, ischemic heart failure, and myocarditis comprising the steps of:
contacting cardiomyocytes having increased Epac activity as compared to normal cardiomyocytes with compounds to screen,
assessing the hypertrophic properties of said cardiomyocytes having increased Epac activity,
selecting compounds which inhibit the hypertrophic properties of said cardiomyocytes having increased Epac activity, as compared to the hypertrophic properties of cardiomyocytes having increased Epac activity not treated by said compounds.

In a further embodiment, the screening method involves the step of:

a) providing cardiomyocytes that express an activated form of Epac
b) exposing said cardiomyocyte to one of the compounds to be tested
c) assessing the effects of said compound on the hypetrophic properties of said cardiomyocytes, the hypertrophic properties being determined by measuring cell size and/or the expression of mRNA coding for hypertrophic gene markers, such as the natriuretic factor.

The cardiomyocytes that have an increased Epac activity as compared to normal cardiomyocytes express for example a wild type form of Epac or a constitutive activated form of Epac lacking its cAMP binding domain.

The inhibition of the hypertrophic properties indicates that said compound is an antagonist of Epac.

Administration of vectors producing ribozyme, siRNA or antisense oligonucleotides may be administered to cardiac cells or cardiac cell lines by any method such as, without limitation, transfection, electroporation, lipofection, and transduction.

In a further embodiment, the screening method can result in the combination of the two above described methods.

The invention further relates to the method as defined above, wherein the compounds to screen are:
cAMP analogues, such as cAMP derivatives or 8-(4-chlorophenylthio)-2′-O-methyladenosine-3′-5-cyclic monophosphate (8-CPT-2′-O-Me-cAMP) derivatives, or
Brefeldin A derivatives.

A further object of the invention resides in a method for screening a compound, and especially a cAMP analog, that affects the activation of Epac.

According to another embodiment, the present invention relates to a non-human transgenic mammal for use as a model of cardiac hypertrophy, wherein Epac activity is increased with respect the corresponding wild type nonhuman mammal.

In this non human model of cardiac hypertrophy, cardiac hypertrophy has been induced by the specific expression of a positive dominant form of Epac, and especially Epac1 in cardiomyocytes.

The invention relates in particular to a non-human transgenic mammal for use as a model of cardiac hypertrophy as defined above, wherein Epac is over-expressed with respect the corresponding wild type non-human mammal, in particular said non-human transgenic mammal comprises Epac coding sequences under the control of cardiac-specific promoters having a stronger transcription activity than Epac natural promoter, such as the promoter of the α-Myosin Heavy Chain.

The positive dominant form of Epac is under the control of a cardiac specific promoter such as α-Myosin Heavy Chain.

The invention further relates to a non-human transgenic mammal for use as a model of cardiac hypertrophy as defined above, wherein said non-human transgenic mammal comprises nucleic sequences encoding a constitutively activated form of Epac lacking the activating cAMP binding domain.

In a further embodiment the positive dominant form of Epac1 results in Epac-ΔcAMP which contains a deletion of the first 322 amino acids of Epac1 (de Rooij et al., 1998). Epac-ΔcAMP lacks the cAMP-binding domain and therefore behaves as a constitutive activated form of Epac and cannot be regulated by cAMP (de Rooij et al., 1998).

The invention also relates to a nonhuman transgenic mammal for use as a model of cardiac hypertrophy as defined above, wherein said mammal is a mouse.

### Therapeutic Methods

The compositions containing the Epac antagonist(s) can be administered for prophylactic and/or therapeutic treatments. The active ingredient in the pharmaceutical composition generally is present in an “effective amount”. By an “effective amount” of a pharmaceutical composition is meant a sufficient, but nontoxic amount of the agent to provide the desired effect. The term refers to an amount sufficient to treat a subject (e.g., a mammal, particularly a human). Thus, the term “therapeutic amount” refers to an amount sufficient to remedy a disease state or symptoms, by preventing, hindering, retarding or reversing the progression of cardiac hypertrophy or any other undesirable symptoms whatsoever. The term “prophylactically effective” amount refers to an amount given to a subject that does not yet present the symptoms of cardiac hypertrophy, and thus is an amount effective to prevent, hinder or retard the onset of cardiac hypertrophy.

The symptoms of cardiac hypertrophy include shortness of breath on exertion, dizziness, fainting and angina pectoris. (Angina is chest pain or discomfort caused by reduced blood supply to the heart muscle.) Some people have cardiac arrhythmias. These are abnormal heart rhythms that in some cases can lead to sudden death. The obstruction to blood flow from the left ventricle increases the ventricle's work, and a heart murmur may be heard. In the majority of patients with hypertrophic cardiomyopathy, the physical examination is unremarkable and the abnormalities may be subtle. Most patients have forceful or jerky pulse and a forceful heart beat, which can be felt on the left side of the chest. Both of these reflect the thickened, strongly contracting heart. However the most obvious abnormality on physical examination is a heart murmur, which is present in 30-40% of patients. To diagnose hypertrophic cardiomyopathy, the electrocardiogram (ECG) is usually used. ECG usually shows an abnormal electrical signal due to muscle thickening and disorganization of the muscle structure. In a minority of patients (approximately 10%) the ECG may be normal or show only minor changes. As MRI (Magnetic Resonance Imaging) can provide tomographic high resolution pictures of the heart, it has recently become an important new test well suited for the assessment of the size and extent of left ventricular hypertrophy in cardiac hypertrophy. In fact, recent studies have shown that a cardiac MRI may be better than an echocardiogram to reliably detect hypertrophy in areas such as the left ventricular anterolateral wall and apex. As a result, in some patients an echocardiogram may not be sufficient to confidently exclude a diagnosis of cardiac hypertrophy and in that situation a cardiac MRI may be recommended.

In therapeutic applications, compositions are administered to a patient already suffering from a disease, as just described, in an amount sufficient to cure or at least partially arrest the symptoms of the disease and its complications. An appropriate dosage of the pharmaceutical composition is readily determined according to any one of several well-established protocols. For example, animal studies (e.g., mice, rats) are commonly used to determine the maximal tolerable dose of the bioactive agent per kilogram of weight. In general, at least one of the animal species tested is mammalian. The results from the animal studies can be extrapolated to determine doses for use in other species, such as humans for example. What constitutes an effective dose also depends on the nature and severity of the disease or condition, and on the general state of the patient's health.

In prophylactic applications, compositions containing at least one Epac antagonists are administered to a patient susceptible to or otherwise at risk of a cardiac hypertrophy. Such an amount is defined to be a “prophylactically effective” amount or dose. In this use, the precise amounts again depend on the patient's state of health and weight.

The following examples are provided to illustrate certain aspects of the methods and compositions that are provided but should not be construed to limit the scope of the claimed invention.

### DESCRIPTION OF THE FIGURES

FIG. 1A, FIG. 1B and FIG. 1C

Epac activates the small G protein Rac in primary rat ventricular cardiomyocytes and HL-1 atria cells.

FIG. 1A: Primary rat ventricular cardiomyocytes were infected with either Ad.GFP as a control or with Ad.EpacWT or Ad.Epac-ΔcAMP as described in Methods. Two days after infection, cells were treated or not for 10 min with the selective activator of Epac, 8-CPT (1 μM). Amounts of Rac-GTP were determined by pull down experiments. A control for total Rac expression (total lysates) is shown. The upper panel shows a typical immunoblot. Expression of recombinant proteins was determined by Western blot using an anti-HA antibody. The lower panel shows means±S.E.M. of 3 independent experiments. Results are expressed as fold activation of control cells infected with Ad.GFP. *p<0.05, **p<0.01 compared with control.

FIG. 1B: HL-1 atrial cells were treated with 8-CPT (100 μM), Forskolin (FSK) (100 μM), or 8-Br-cAMP (10 μM) for 10 min. The upper panel shows a representative immunoblot. In lower panel, Rac-GTP is expressed as fold activation of the control (CT) cells (means±S.E.M; n=4).

FIG. 1C: Effect of 8-CPT (10 μM) at different time of incubation on the amount of Rac-GTP. Rac activation was determined as above. Control for total lysates (Total Rac) is shown.

FIG. 2A, FIG. 2B, FIG. 2C and FIG. 2D

Epac stimulates a hypertrophic pattern of gene expression.

FIGS. 2A, 2C and 2D: Neonatal cardiomycoytes were transfected with ANF-Luc (FIG. 2A), SkM-α-actin-Luc (FIG. 2C) or c-fos-SRE-Luc (FIG. 2D) and EpacWT, RacG12V or the empty vector (mock) as control and treated or not with 8-CPT (1 μM). Two days after transfection, cells were assayed for luciferase activity. Results are expressed as percentage activation of control. Results are means±S.E.M. for 3 independent experiments performed in triplicates.

FIG. 2B: Epac induces expression of ANF mRNA. Cardiomyocytes were infected with Ad.EpacWT, Ad.RacG12V, or Ad.GFF (control) and stimulated or not with 8-CPT (1 μM) for 2 days. ANF mRNA expression was determined by quantitative PCR as described in Methods. Values are expressed relative to the ANF/GCB ratio and results were normalized to control for each experiment. Results are presented as the mean±S.E.M. of 3 independent experiments performed in duplicates. *p<0.05, **p<0.01, ***p<0.001 compared with control.

FIG. 3A, FIG. 3B, FIG. 3C, FIG. 3D and FIG. 3E

Epac induces cardiomyocytes hypertrophy in neonatal (FIGS. 3A, 3B and 3C) and adult (FIGS. 3D and 3E) rat primary cardiomyocytes.

FIG. 3A: Fluorescent microscopic analyses of the effects of, Epac on sarcomeric organization. Morphology of representative myocytes 48 h after infection with Ad.GFP as a control, or Ad.EpacWT is shown. The Epac selective activator, 8-CPT, was used at 1 μM for 2 days in cells infected with Ad.GFP. For a positive control, cells were infected with Ad.GFP and treated with PE (1 μM) for 2 days. Actin filaments were visualized by using Rhodamin-conjugated phalloidin.

FIG. 3B: Photographic images of cells infected for 2 days with Ad.GFP or Ad.EpacWT and treated or not with either 8-CPT (1 μM) or PE (1 μM) were digitised. The areas (μm²) of 30 to 50 individualized cells per condition from 2 to 3 independent experiments were determined by computer-assisted planimetry. Values show the means±S.E.M.

FIG. 3C: [³H]-leucine incorporation. Cardiomyocytes were treated as above and total radioactivity of incorporated [³H]-leucine into proteins was determined by scintillation counting. The figure shows the mean±S.E.M. of data for 3 experiments performed in duplicate. *p<0.05, **p<0.01, ***p<0.001 compared with control Ad.GFP.

FIG. 3D: α-actinin staining of cardiac myocytes infected with the Ad.GFP or the Ad.Epac^{WT} and GFP, or Ad.Epac^{dcAMP}, or Ad.Rap^{Q63E}, treated or not with an agonist of Epac, 8-CPT. Morphology of representative myocytes 48 h after infection with Ad.GFP as a control, Ad.Epac^{WT}/GFP or Ad.Rap^{Q63E} is shown. The Epac selective activator, 8-CPT was used at 1 μM for 2 days. α-actinin was visualized by immunocytochemistry as described in Methods. Pictures show from the upper to the lower panel: α-actinin staining (upper panel), GFP expression (middle), and merge from the two previous pictures (lower panel).

FIG. 3E: Photographic images of rat adult cardiac myocytes infected for 2 days with the Ad.GFP, or Ad.Epac^{WT}, or Ad.Epac^{dcAMP} or Ad.Rap^{Q63E} and treated or not with 8-CPT (1 μM) and were digitized. The surface, the length, and the width of around 100 individualized cells per condition from 5 to 6 independent experiments were determined by computer-assisted planimetry. Values show the means±S.E.M.

FIG. 4A, FIG. 4B, FIG. 4C, FIG. 4D and FIG. 4E

Epac induces intracellular Ca2+ transients and Ca2+ activates Rac.

In FIGS. 4A, 4B, 4C and 4D, neonatal cardiomyocytes at day 1 or 2 after plating were loaded with the Ca2+ indicator Fluo3-AM and perfused with a control external ringer solution.

FIG. 4A: Effect of 10 μM 8-pCPT-2′-O-Me-cAMP (8-CPT) on spontaneous spiking activity at 1.8 mM external [Ca²⁺].

FIG. 4B: Effect of 10 μM 8-CPT in the presence of 20 mM external Cs⁺.

FIG. 4C: Effect of 100 μM 8-CPT in the absence of external Ca²⁺.

FIG. 4D: Effect of 100 μM 8-CPT in the absence of external Ca²⁺ and presence of the PKA blocker H89 (1 μM).

FIG. 4E: Effect of ionomycin on Rac activation. Primary rat ventricular cardiomyocytes were treated at 2 days in vitro with ionomycin (1 μM) for different times of incubation or PE (1 μM) for 15 min as a positive control. The amount of Rac-GTP was determined by pull down experiments followed by Western blotting using an anti-Rac antibody.

FIG. 5A, FIG. 5B and FIG. 5C

Activation of NFAT by Epac.

FIG. 5A and FIG. 5B: Effect of Epac on NFAT transcriptional activity. Cardiomyocytes infected with Ad.GFP (control), Ad.EpacWT, or Ad.VIVIT were transfected with NFAT-Luc and treated or not with CsA (0.5 μM) for 48 h. Luc activity was assayed as described in Methods.

FIG. 5C: Epac increases MCIP1 expression. Cardiac myocytes infected with Ad.GFP (control) or Ad.EpacWT were treated or not with 8-CPT (1 μM) for 2 days. The ratio of MCIP1/GCB mRNA was determined by quantitative PCR. Values are expressed relative to the MCIP1/GCB ratio.

Results were normalized to control for each experiment, and were expressed as means±S.E.M of at least 3 independent experiments performed in triplicate (FIGS. 5A and 5B) or duplicate (FIG. 5C).

FIG. 6A and FIG. 6B

Ad.VIVIT inhibits Epac-induced cardiomyocyte hypertrophy

FIG. 6A: Fluorescent microscopic analyses of the effects of Epac on sarcomeric organization. Morphology of representative myocytes 48 h after infection with Ad.GFP as a control, Ad.VIVIT, Ad.EpacWT, or Ad.EpacWT and Ad.VIVIT is shown.

FIG. 6B: Photographic images of cardiac myocytes infected as above were digitised. Areas (μm²) of around 50 individualized cells per condition from 3 independent experiments were determined by computer-assisted planimetry. Values show the means±S.E.M. *p<0.05, **p<0.01 and ***p<0.001 compared with control or versus indicated values.

FIG. 7A, FIG. 7B and FIG. 7C

Epac activates CaMKII signaling pathway.

FIG. 7A: Epac regulates MEF2 transcriptional activity. Cardiomyocytes infected with Ad.GFP (control), Ad.EpacWT, or Ad.EpacΔcAMP were transfected with MEF2-Luc and treated or not with CsA (0.5 μM) for 48 h. Luc activity was assayed as described in Methods.

FIG. 7B: Epac regulates MEF2 transcriptional activity. Cardiomyocytes infected with Ad.GFP (control), Ad.EpacWT, or Ad.EpacΔcAMP were transfected with MEF2-Luc and treated or not with KN-93 (1 μM) for 48 h. Luc activity was assayed as described in Methods.

FIG. 7C: KN-93 inhibits Epac-induced cardiomyocyte hypertrophy. Cardiomyocytes infected with Ad.GFP (control), or Ad.EpacΔcAMP and treated or not with KN-93 (1 μM) for 2 days were stained with phalloidin. Photographic images were then taken and digitised. Areas (μm²) of around 50 individualized cells per condition from 3 independent experiments were determined by computer-assisted planimetry. Results were normalized to control for each experiment, and were expressed as means±S.E.M of at least 3 separate experiments performed in triplicate.

FIG. 8A, FIG. 8B and FIG. 8C

Involvement of Rac in Epac-induced NFAT dependent cardiomyocyte hypertrophy.

In FIGS. 8A, 8B and 8C, cardiomyocytes infected with Ad.GFP (control), Ad.RacS17N, Ad.EpacWT, or Ad.EpacWT and Ad.RacS17N were transfected with NFAT-Luc, MEF2-Luc or ANF-Luc. Two days after, Luc activity was determined. Luc activity was normalized to control for each experiment, and were expressed as means±S.E.M of at least 3 separate experiments performed in triplicate

In FIG. 8B, lower panel, the expression of the infected constructs was monitored using antibodies directed against HA and c-Myc.

FIG. 9A and FIG. 9B

Ad.RacS17N reverses Epac-induced cardiomyocyte hypertrophy.

FIG. 9A: Photographic images of cells infected for 2 days with Ad.GFP (control), Ad.EpacWT, Ad.RacS17N, or Ad.EpacWT and Ad.RacS17N were digitised.

FIG. 9B: The areas (μm²) of 50 individualized cells per condition from 3 independent experiments were determined by computer-assisted planimetry. Values show the means±S.E.M. *p<0.05, **p<0.01 and *** p<0.001 versus control cells or indicated values.

FIG. 10

Possible hypertrophic signaling pathways controlled by Epac.

Stimulation of Gs-coupled receptor activates the adenylyl cyclase (AC) which increases intracellular cAMP levels. Subsequent activation of Epac induces a rise in [Ca2+]i which increases Rac activation. The small G protein Rac can both induce cytoskeletal reorganization and activation of the calcineurin/NFAT signaling pathway. Epac also regulates MEF2 transcriptional activity via CaMKII. Epac signaling pathway induces hypertrophic gene expression and cardiomyocyte growth.

FIG. 11

Activation of Epac 1 increases protein synthesis in adult rat primary cardiomyocyte.

Cardiomyocytes were infected with the Ad.GFP (control), or the Ad.Epac^{WT} and GFP, or Ad.Rap^{Q63E}, treated or not with an agonist of Epac, 8-CPT, and total radioactivity of incorporated [3H]-leucine into proteins was determined by scintillation counting. The Epac selective activator, 8-CPT was used at 1 μM for 2 days. As a positive control, cells were treated with the hypertrophic stimulus, phenylephrine for 2 days (1 μM). The figure shows the mean±S.E.M. of data for 3 experiments performed in duplicate. *p<0.05, **p<0.01, compared with control Ad.GFP.

FIG. 12

Epac1 is increased in patients with heart failure (HF, n—22).

Change in gene expression was analyzed by quantitative RT-PCR. Histograms show the relative mRNA abundance of human Epac1. Data are expressed as fold over control patients (n=11) normalized to the housekeeping gene GCB. Results are mean values±SEM.

FIG. 13

Schematic representation of a bicistronic adenovirus (Ad.) bearing either Epac^{WT} (wild type) or Epac^{ΔcAMP} (constitutive activated form of Epac1) under the control of a cytomegalovirus (CMV) promoter, and green fluorescent protein (GFP) under internal ribosomal entry site (IRES) control.

FIG. 14

Schematic representation of the α-MHC-HA-EpacΔAMP.

The cDNA coding for the human form of Epac1 lacking its first 322 amino acids (EpacΔcAMP) and containing a HA epitope (HA) in its N terminus was fused upstream the α-Myosin Heavy Chain cardiac specific promoter (α-MHC).

### EXAMPLES

### Example 1

### The cAMP-Binding Protein Epac Induces Cardiomyocyte Hypertrophy

### Materials and Methods

### Materials

All media, sera and antibiotics used in the cell culture were purchased from Invitrogen (Cergy Pontoise, France). 8-(4-chloro-phenylthio)-2′-O-methyladenosine-3′-5′cyclic monophosphate (8-pCPT-2′-O-Me-cAMP) was from Biolog Life Science Institute (Bremen, Germany). Forskolin, 8-Bromo-cAMP, Phenylephrine and H89 were obtained from Calbiochem (France Biochem, Meudon, France).

### Cell Culture

HL-1 atrial cardiomyocytes, a gift from Dr. Claycomb (Louisiana State University, New Orleans, La., U.S.A.) were plated onto fibronectin-gelatin-coated plates or coverslips and cultured in Claycomb medium supplemented with 10% fetal bovine serum, 100 units/ml penicillin, 100 μg/ml streptomycin, 0.1 mM norepinephrine, and 2 mM L-glutamine as described (Claycomb et al, 1998).

Neonatal rat ventricular myocytes were isolated according to the protocol described by Wollert and colleagues (Wollert et al. 1996) and modified as follows: ventricular cells from one day old wistar rats were individualized by digestion with collagenase A (Roche Diagnostics Corporation, Germany) and pancreatin. The cell suspension was purified by centrifugation through a discontinuous Percoll gradient (Sigma Aldrich, L'Isle d'Abeau Chesmes, France). Cardiomyocytes were plated in gelatin-coated culture dishes in Dubelcco's modified Eagle's medium (DMEM)/medium 199 (4/1) supplemented with 10% horse serum (v/v), 5% newborn calf serum (v/v), glutamine and antibiotics. The day after, cardiomyocytes were switched to the maintenance medium composed of DMEM/medium 199 supplemented only with glutamine and antibiotics.

Adult rat ventricular myocytes were isolated from male Wistar rats (160-180 g). The rats were subjected to anesthesia by intraperitoneal injection of pentothal (0.1 mg/g), and hearts were excised rapidly. Individual ventricular myocytes were obtained by retrograde perfusion of the heart as previously described (Verde et al., 1999). Freshly isolated cells were suspended in minimal essential medium (MEM: M 4780; Sigma) containing 1.2 mM Ca²⁺, 2.5% fetal bovine serum (FBS, Invitrogen, Cergy-Pontoise, France), 1% penicillin-streptomycin and 2% HEPES (pH 7.6) and plated on laminin-coated culture dishes (10 μg/ml laminin, 2 h) at a density of 10⁵ cells per dish. The cells were left to adhere for 1 h in a 95% O₂, 5% CO₂ incubator at 37° C. before adenoviral infection.

### Adenoviral Infection

Bicistronic adenoviruses (Ad5) bearing either EpacWT or EpacΔcAMP under the control of a cytomegalovirus (CMV) promoter, and green fluorescent protein (GFP) under internal ribosomal entry site (IRES) control were constructed and amplified at the Genethon Center of Evry (France) (FIG. 13). Adenoviruses encoding VIVIT, a selective peptide inhibitor of calcineurin-mediated NFAT activation, and Rac were provided by Drs. J. Molkentin and T. Finkel, respectively. One day after plating, cardiomyocytes were incubated for 2 h with recombinant adenoviruses. After removal of the virus suspension, cells were replaced in maintenance medium for 2 days and then stimulated with the different drugs. Viruses were used at a multiplicity of infection (MOI) of 100.

Concerning the adenoviral infection of adult rat ventricular myocytes, the cells were left to adhere for 1 h in a 95% O₂, 5% CO₂ incubator at 37° C., before the medium was replaced by 200 μl of Fetal Bovine Serum (FBS)-free MEM containing the Epac1 wild type (WT)-encoding adenovirus (Ad.Epac^{WT}), the constitutive active form of Epac1 (Ad.EpacΔcAMP) adenovirus, the constitutive active form of Rap1 (Ad.Rap1^{Q63E}) or the green fluorescent protein-encoding adenovirus (Ad.GFP). Ad.Epac^{WT}, Ad.Rap1^{Q63E} or Ad. GFP were used at a multiplicity of infection (MOI) of 100 plaque-forming units (pfu) per cell, whereas Ad.EpacΔcAMP was generally used at 500 MOI (see results). After 2 h, the same volume of FBS-free medium without adenovirus was added, and the cells were placed for 2 days in an incubator (37° C., 5% CO2). The medium was changed the next morning for adenovirus- and FBS-free MEM.

### Plasmid Constructs and Transfection

The plasmid constructs were generously provided by the following: a −3003 bp fragment of the rat ANF promoter fused to the luciferase reporter gene (ANF-Luc) by Dr. K. Knowlton, Luciferase reporter genes linked to promoters for skeletal muscle α-actin (SkM-α-actin-Luc) and serum response element-regulated c-fos (c-fos-SRE-Luc) by Dr. M. D. Schneider, Epac1 plasmid constructs by Dr. J. Bos. The luciferase reporter plasmid driven by four NFAT consensus binding sites (NFAT-Luc) or driven by the three MEF2 consensus binding sites (MEF2-Luc) was obtained from Stratagene and was kindly provided by Dr K C Wollert respectively. Transient transfection experiments were performed with Lipofectamine 2000 (Invitrogen Life Technologies, France) in optimem medium in the presence of 1 mg of the various plasmid constructs according to the manufacturer's instructions. Two days post-transfection, cells were lysed and assayed at 37° C. for Luciferase activity using a Luciferase assay kit (promega corporation, Madison USA) in conjunction with a luminometer allowing automated luciferin solutions injection (Lumat LB 9507, EG & G Berthold).

### [3H]-Leucine Incorporation

The assessment of protein synthesis was achieved by adding 1 mCi/ml of [3H]-leucine (Amersham Chemical Corp.) to each well for 24 h (neonatal cardiomyocytes) or 48 h (adult cardiomyocytes) in presence of adenoviruses and/or in stimulated conditions in the maintenance medium. Thereafter, the [3H]-leucine-containing medium was aspirated. Myocytes were washed three times with phosphate-buffered saline (PBS) and incubated with 5% trichloroacetic acid for 30 min at 4° C. The cell residues were rinsed in 70% and then 100% ethanol, solubilized in 0.33 M NaOH for 1 h and scrapped with a rubber policeman. Radioactivity was measured in a liquid scintillation counter (LS 6000 SC Beckman).

### Actin Staining and Surface Area

Cardiomyocytes were plated in Lab-Tek plastic chamber slides (Nunc), precoated with gelatin (0.2%) and incubated in the presence or absence of various agents. After 48 h, cells were rinsed three times with PBS containing 1% BSA and fixed by immersion in 4% paraformaldehyde during 30 min.

For actin cytoskeletal organization, cardiomyocytes were incubated with a 1/800 diluted solution of rhodamine phalloidine (Sigma Aldrich, L'isle d'Abeau Chesmes, France) for 45 min with gentle shaking.

Alternatively, cardiac actin organization was assessed by the following protocol: myocytes were incubated with 0.2% Triton X-100 for 5 min, followed by 0.5 mol/L NH₄Cl in PBS for 15 min. After a preincubation in 5% bovine serum albumin in PBS for 30 min, myocytes were incubated overnight with a mouse monoclonal antibody directed against cardiac sarcomeric α-actinin (cloneEA-53, 1/300, Sigma, France). The secondary antibody used was a goat anti-mouse IgG coupled to Alexa Fluor®594 (1/150, Molecular Probes).

Following washing with PBS, cells were finally mounted on glass coverslips in mowiol antifadent mounting medium (mowiol 8%, glycerol 8%, DABCO (di-aza-bicyclo-octane); France Biochem, Meudon, France). F-actin was analysed by confocal scanning laser microscope. Optical section series were obtained with a Plan Apochromat 63× objective (NA 1.4, oil immersion). Morphometric parameters were determined by computer-assisted planimetry (Perkin Elmer). Thirty to fifty individualized cells using the rhodamine phalloidine and 500 to 600 individualized cells using the cardiac sarcomeric α-actinin antibody were analyzed for each condition.

### Rac Activation Assay

Rac pull-down experiments were performed using a GST fusion protein containing the Cdc42/Rac Interactive Binding Domain (CRIB) of PAK as previously described (Maillet et al, 2003). After stimulation, cells were lysed in RIPA buffer (50 mM Tris-HCl, pH 7.5; 500 mM NaCl; 20 mM MgCl2; 0.5% deoxycholic acid; 0.1% SDS; 1% Triton X-100; 1 mM PMSF; 10 mg/ml leupeptin and aprotinin) and 2 mg of protein were incubated with GST-CRIB coupled to glutathione-sepharose beads (Amersham Biosciences) for 1 h at 4° C. Beads were then washed three times in Rac washing buffer (50 mM Tris-HCl, pH 7.5; 150 mM NaCl, 20 mM MgCl2, 1% Triton X-100; 0.1 mM PMSF; 10 mg/ml leupeptin and aprotinin). Rac-GTP samples and total lysates were separated on SDS-PAGE gels and transferred onto a polyvinylidene difluoride (PVDF) membrane (Amersham Pharmacia Biotech). Membranes were hybridised with an anti Rac1 monoclonal antibody (Upstate Biotechnology) and proteins were revealed by enhanced chemiluminescence (ECL+; Amersham Pharmacia Biotech).

### Intracellular Ca2+ Measurements

Neonatal rat ventricular myocytes at day 1 to 2 after isolation were loaded with the Ca2+ indicator Fluo 3 AM (Molecular Probes, 10 μM, 30 min, 37° C.) in serum-free maintenance medium, and then washed for additional 30 min in external ringer solution containing (nm) NaCl 121, KCl 5.4, Hepes 10, Glucose 5, Na-pyruvate 5, NaHCO3 4, Na2HPO4 0.8, MgCl2 1.8, CaCl2 1.8. Experiments were carried out in this solution or in the ringer solution with 100 μM EGTA and without added Ca2+ and Mg2+ and. In order to block HCN channels, 20 mM CsCl was added to the initial Ca2+ solution, while KCl was omitted and NaCl was reduced to 107 mM to maintain osmolarity constant. Cells were mounted on the stage of an inverted Nikon Eclipse microscope and visualized using a 63× oil immersion fluorescence objective. The field was illuminated at 488 nm with a xenon lamp. Images at 535 nm were captured by a CCD camera (Sensicam QE, photoline) driven by Metafluor software. Experiments were performed at room temperature.

### Reverse Transcription-Polymerase Chain Reaction (RT-PCR)

Left-ventricular samples obtained from human hearts were classified into two groups, control (“normal”) donors and end-stage failing hearts. Total RNA was prepared from human tissue or from neonatal rat cardiomyocytes using the Trizol RNA purification system (Life Technologies Inc.). RNA was then treated with DNase I (Life Technologies Inc.) and 3 mg of total RNA were then hybridized with oligo(dT) primer and reverse transcribed using Superscript reverse transcriptase II (Life Technologies Inc.). Quantitative RT-PCR was conducted with a LightCycler system (Roche) using a LightCycler-FastStart DNA Master SYBR Green I kit (Roche) and specific primer pair for human Epac1 (5′-GCTCTTTGAACCACACAGCA-3′; 5′-TGTCTTCTCGCAGGATGATG-3′), or ANF (5′-GGGCTCCTTCTCCATCACCAA-3′; 5-CTTCATCGGTCTGCTCGCTCA-3′) or MCIP1-(5′-AGCGAAAGTGAGACCAGGGC-3′; 5′-GGCAGGGGGAGAGATGAGAA-3′). PCR reactions were performed using the following cycle conditions: denaturation for 10 sec at 95° C., annealing for 5 s at 60° C. and extension for 11 sec at 72° C. Dissociation curves were generated after each PCR run to ensure that a single specific product was amplified. Glucocerebrosidase (GCB) was measured as a reference gene using the primer pair 5′-GCACAACTTCAGCCTCCCAGA-3′ and 5′-CTTCCCATTCACCGCTCCATT-3′.

### Statistical Analysis

Results are expressed as means±SEM. Differences between groups have been analyzed by one-way ANOVA followed by unpaired Student's t test. Differences were considered significant when * P<0.05, ** P<0.01, *** P<0.001.

### Results

### Epac Activates the Small G Protein Rac in Cardiomyocytes

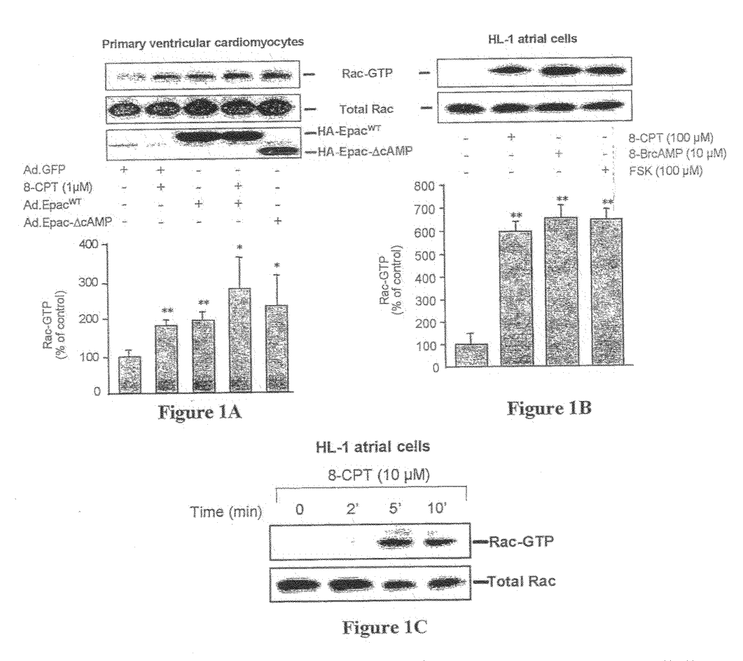

The Inventors directly assayed Rac GTP-loading using a glutathione S-transferase (GST) fusion protein containing the Cdc42-Rac interactive binding domain (CRIB) of p21-activated kinase (PAIL). Activation of endogenous Epac with a selective activator of this GEF, 8-pCPT-2′-O-Me-cAMP (8-CPT) (Enserink et al, 2002) increased Rac activation in rat cardiac myocytes (FIG. 1A). Similarly, infection of cardiomyocytes with an adenovirus encoding Epac1WT (Ad.EpacWT) significantly enhanced Rac GTP-loading compared to control cells infected with GFP (FIG. 1A). Rac activation was further increased when cells infected with Ad.EpacWT were treated with 8-CPT (1 μM) for 10 min (FIG. 1A). As shown in FIG. 1A, an adenovirus bearing a constitutive activated form of Epac1 (Ad.Epac-ΔcAMP) (de Rooij et al, 1998) significantly induced Rac activation. The fact that Ad.EpacWT had similar effects on Rac activity as observed for Ad.Epac-ΔcAMP could be explained by its activation with endogenous cAMP. The regulation of Rac activity by Epac was not restricted to ventricular cardiomyocytes since a 10 min exposure of HL-1 adult mouse atrial cells to 8-CPT (100 μM) caused a strong increase in the amount of Rac-GTP (FIG. 1B). In this cell line, 8-CPT-induced Rac activation was detected upon 5 min treatment with this cAMP analogue (FIG. 1C). Furthermore, the Inventors observed that a potent activator of adenylyl cyclase, forskolin (100 μM) and a cAMP analogue, 8-Br-cAMP (100 μM) mimicked the effect of 8-CPT on Rac activation (FIG. 1B). Altogether, these results demonstrate that recombinant and native Epac induce the conversion of Rac into an activated state in atrial and ventricular cardiomyocytes.

### Epac Increases the Expression of Hypertrophy Gene Markers

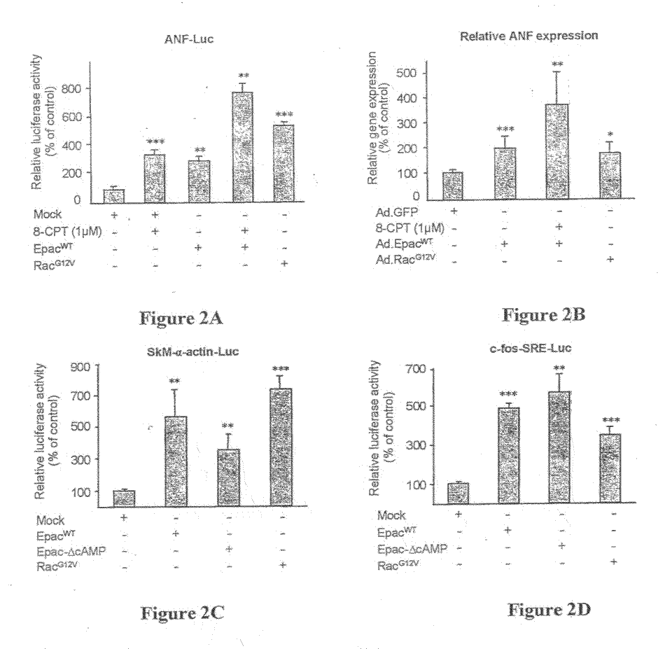

As Rac has been found to be involved in cardiac myocyte hypertrophy (Pracyk et al, 1998; Sussman et al, 2000), the Inventors next tested the potential involvement of Epac in this process. Re-expression of embryonic genes and transient activation of immediate early genes are frequently used indexes of myocyte hypertrophy (Chien et al, 1991). The ability of Epac to stimulate gene expression was determined using luciferase (Luc) constructs under the control of promoters for atrial natriuretic factor (ANF), skeletal muscle (SkM) α-actin and the c-fos-responsive element (c-fos-SRE). FIG. 2A shows a three fold activation of the ANF-Luc reporter gene in neonatal cardiomyocytes stimulated with 8-CPT (1 μM) compared to control cells. Transient transfection of Epac1WT as well as 8-CPT (1 μM) increased the basal level of ANF-Luc activity (FIG. 2A). A constitutive activated form of Rac (RacG12V) mimicked the effect of Epac on ANF-Luc activity (FIG. 2A). Accordingly, endogenous expression of ANF mRNA was significantly increased in ventricular cardiomyocytes infected with Ad.EpacWT and stimulated or not with 8-CPT (1 μM), as compared to cell infected with control Ad.GFP (FIG. 2B). Similar results were obtained with an adenovirus expressing Ad.RacG12V (FIG. 2B). In addition, when cotransfection experiments were performed with SkM-α-actin-Luc or c-fos-SRE-Luc, it was found that EpacWT, Epac-ΔcAMP, or RacG12V significantly increased Luc activity compared to control cells (FIGS. 2C and 2D).

### Epac Increases Cardiomyocyte Size and Sarcomeric Organization

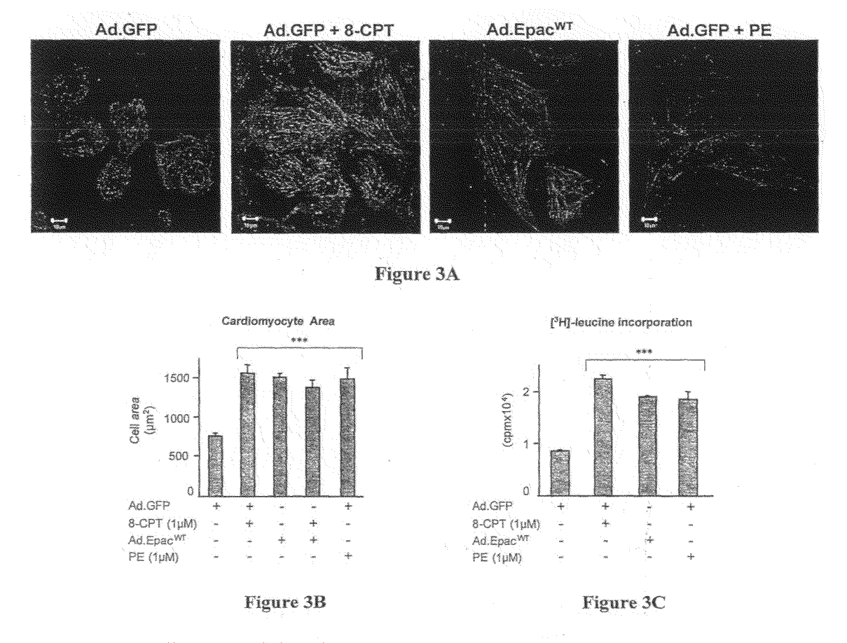

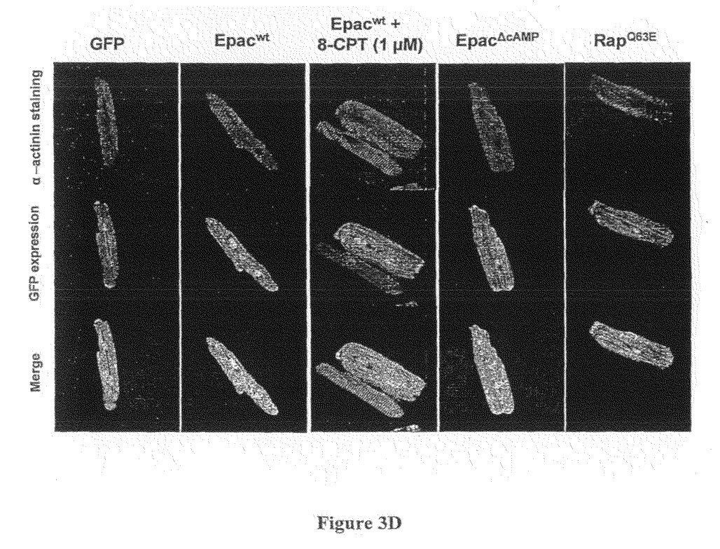

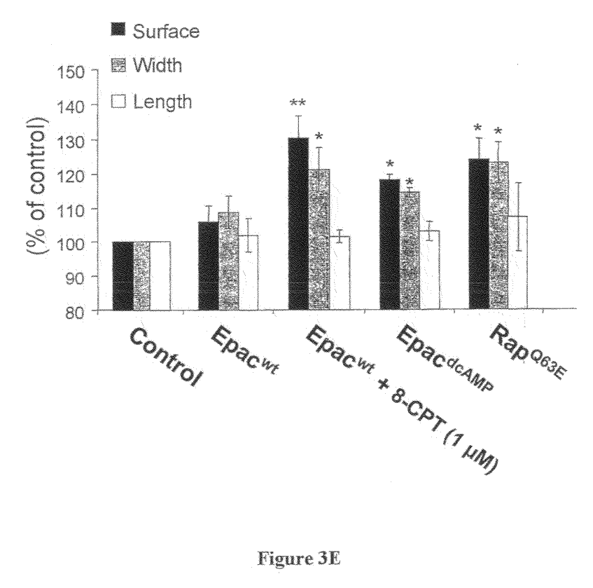

Further studies were undertaken to determine the effects of Epac on other features of the hypertrophic program such as cell size and sarcomeric organization. Cytoskeletal organization was analysed by phalloidin staining. Cardiomyocyte treatment with Ad.GFP and 8-CPT (1 mM) as well as infection of cardiomyocytes with Ad.EpacWT induced an apparent increase of the F-actin meshwork and an heavily striated appearance, reflecting the organization of this F-actin cytoskeleton into sarcomeric structures, as compared to cardiomyocytes infected with Ad.GFP alone (FIG. 3A). The effects of Epac on sarcomeric organization were comparable to Ad.Epac-ΔcAMP (data not shown) and PE (1 mM) (FIG. 3A), a well known inducer of cardiac hypertrophy. To further characterize the ability of Epac to induce morphological hypertrophy, the Inventors measured cell surface area. Activation of endogenous Epac with 8-CPT (1 μM) produced a two fold increase in cell surface area when compared to cardiac myocytes infected with control Ad.GFP (FIG. 3B). Identical results were obtained when cardiomyocytes were infected with Ad.EpacWT (FIG. 3B), Ad.Epac-ΔcAMP (data not shown), or Ad.GFP and treated with PE (1 μM) (FIG. 3B). The effect of Ad.EpacWT on cell surface area was not further increased in the presence of 8-CPT (1 μM) suggesting that intracellular cAMP was sufficient to activate recombinant Epac to induce its maximal effect on protein synthesis (FIG. 3B).

Similar results were obtained with adult rat primary cardiomyocytes. The actin organization was assessed using an antibody directed against cardiac sarcomeric α-actinin and then cell surface area was measured. FIGS. 3D and 3E show that cells infected with Ad.EpacWT in the presence of 8-CPT (1 μM), Ad.Epac-ΔcAMP or a constitutive activated form of Rap1, Rap^{Q63E} significantly increased cell size as compared to control cells infected with GFP.

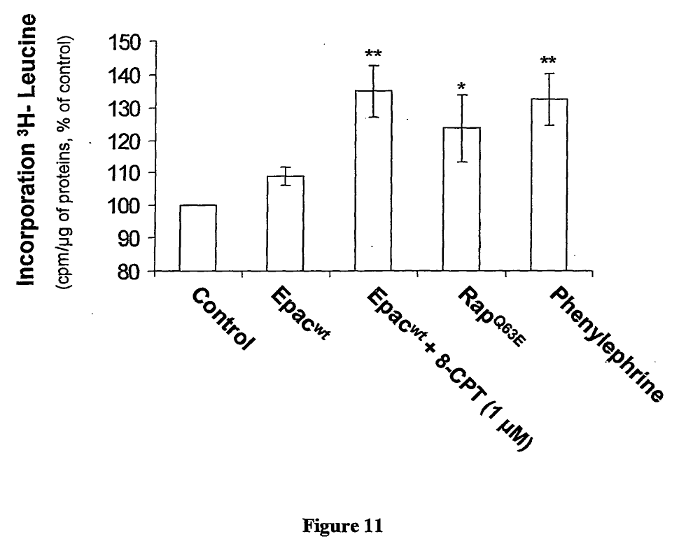

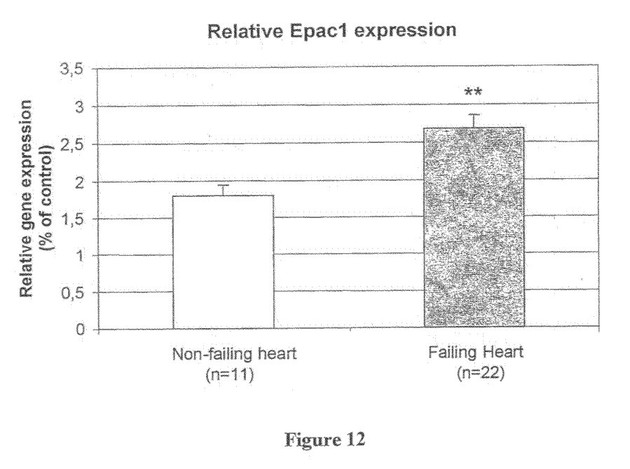

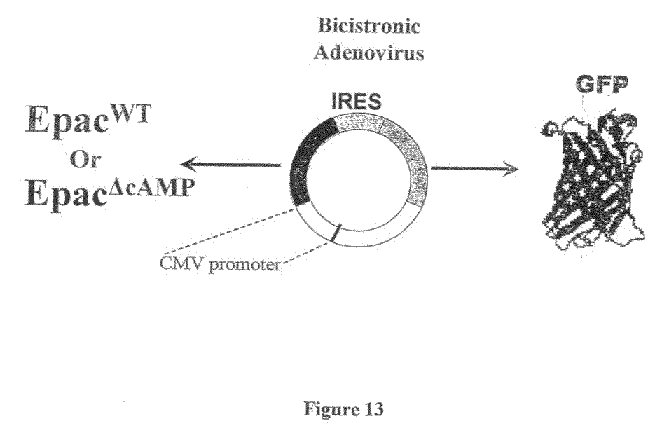

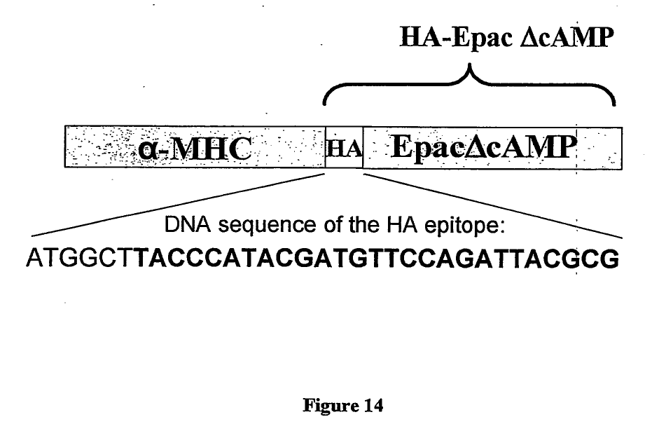

Finally, the effect of Epac on protein synthesis was analyzed by measurement of [3H]-leucine incorporation into cardiac myocytes. Consistent with the ability of Ad.EpacWT to increase cell surface area, expression of this cAMP-GEF resulted in an increase in [3H]-leucine uptake into cardiomyocytes (FIG. 3C). Similarly, cell treatment with the Epac-selective cAMP analogue, 8-CPT (1 μM) or the gold standard, PE (1 μM) resulted in an approximate two fold increase in protein synthesis (FIG. 3C). In addition, Ad.EpacWT in the presence of 8-CPT and Rap^{Q63E} significantly increased protein synthesis as compared to control cells infected with Ad.GFP in primary adult cardiac myocytes (FIG. 11). Altogether, these results show that Epac activation confer all the features of the hypertrophic phenotype in primary ventricular cardiomyocytes.

### Epac Activation Increases Ca2+ Transients Frequency

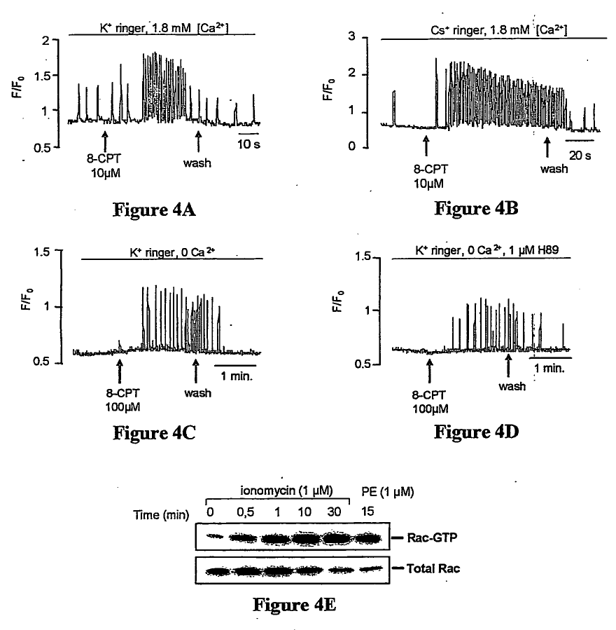

Alterations in intracellular Ca2+ handling progressively exacerbate a hypertrophic or cardiomyopathic phenotype, in part, through sustained activation of Ca2+-sensitive signal transduction pathways (Balke & Shorofsky, 1998). Given the involvement of Epac in cardiac hypertrophy, the Inventors examined whether its activation could affect intracellular Ca2+ concentration ([Ca2+]i) in neonatal myocytes (FIG. 4). As seen in FIG. 4A, at physiological external [Ca2+], these cells exhibited spontaneous Ca2+ transients with a low frequency (0.120±0.015 Hz, n=20). Application of the Epac agonist 8-CPT (10 μM) triggered a dramatic increase in the frequency of these Ca2+ oscillations (0.51±0.04 Hz, n=7) without changing the amplitude of the spikes. This effect was also observed at 100 nM 8-CPT (0.40±0.05 Hz, n=13, data not shown). HCN channels underlie the pacemaker current If which is an important contributor of automaticity in neonatal ventricular cells (Er et al, 2003). If is directly regulated by cAMP and its activation by the cAMP analogue, 8-CPT would be expected to increase spontaneous diastolic depolarisation and rhythmic activity. To test this hypothesis, the Inventors analysed the effect of 8-CPT (10 μM) in the presence of 20 mM Cs+ in the external medium to block HCN channels. As seen in FIG. 4B, external Cs+ failed to prevent the effect of 8-CPT on spontaneous Ca2+ transients, and rather amplified them. Indeed, in these conditions, spike frequency was increased from 0.09±0.11 Hz to 1.25±0.35 Hz by 8-CPT (n=8).

The Inventors next tested the dependence of the 8-CPT effect towards extracellular Ca2+. In the absence of this ion in the bath, spontaneous Ca2+ transients were virtually abolished and 8-CPT (10 μM) had no effect (data not shown). However, increasing the concentration of the Epac agonist to 100 μM triggered the generation of Ca2+ spikes (0.004±0.002 Hz in basal and 0.12±0.02 Hz in the presence of 100 μM 8-CPT, n=4, FIG. 4C). This effect was independent of PKA since an inhibitor of this kinase, H89 failed to block 8-CPT-induced increase in Ca2+ transients frequency (FIG. 4D). In the presence of H89 (1 μM), 8-CPT (100 μM) increased the basal spike frequency from 0.012±0.007 Hz to 0.23±0.049 Hz (n=8). These data indicate that Epac activation produces bursts of Ca2+ transients in neonatal cardiac myocytes, in part by mobilizing an internal Ca2+ source.

As described above, Epac induced Rac activation. Therefore, the Inventors examined the dependence of Rac activation on Ca2+ signaling. Treatment of cardiac myocytes with the Ca2+ ionophore ionomycin (1 μM) increased Rac activation in a time dependent manner (FIG. 4E). The effect of ionomycin on Rac activation was as potent as PE (1 μM) which was used as a positive control in the experiments (FIG. 4). From these results the Inventors conclude that elevation of intracellular [Ca2+]i is sufficient to activate Rac.

### Epac Activates the Hypertrophic Calcineurin/NFAT and MEF2 Signaling Pathways

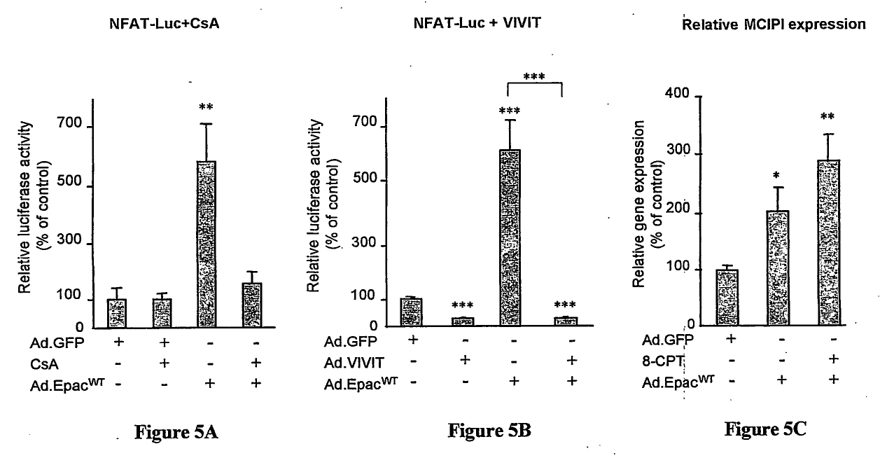

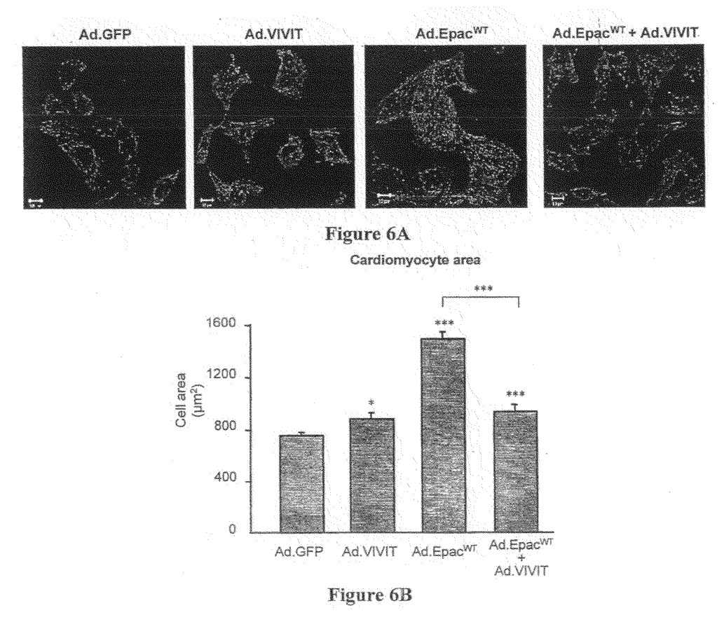

To test whether Epac may activate the hypertrophic calcineurin NFAT signaling pathway, primary cardiomyocytes were transfected with a Luc reporter plasmid driven by four NFAT consensus binding sites (NFAT-Luc) and infected with Ad.EpacWT. As shown in FIG. 5A, Ad.EpacWT significantly increased NFAT transcriptional activity as compared to control cells infected with Ad.GFP. In contrast, Ad.EpacWT had no effect on the promoter-less pGL3 basic vector (data not shown). Epac-induced NFAT transcriptional activity was blocked by a pharmacological inhibitor of calcineurin, cyclosporine A (CsA) (0.5 μM) (FIG. 5A). Consistent with this finding, an adenovirus bearing a selective peptide inhibitor of calcineurin named VIVIT (Ad.VIVIT) (Aramburu et al, 1999), blocked the stimulating effect of Ad.EpacWT on NFAT transcriptional activity (FIG. 5B). In addition, the Inventors found that cardiac myocytes infected with Ad.EpacWT and treated or not with 8-CPT (1 μM) (FIG. 5C), or Ad.Epac-DcAMP (data not shown) had an increased mRNA encoding the modulatory calcineurin-interacting protein 1 (MCIP1), a mediator of calcineurin signaling during cardiac hypertrophy (Yang et al, 2000). The Inventors also analysed the effect of Ad.VIVIT on the Epac-induced cytosketal reorganization into a sarcomeric structure by phalloidin staining. Co-infection with Ad.VIVIT and Ad.EpacWT reduced the enhancement of sarcomeric organization induced by Ad.EpacWT (FIG. 6A). As expected, the ability of Ad.EpacWT to increase cell surface area was significantly decreased by Ad.VIVIT (FIG. 6B). Altogether these data show that NFAT is a downstream component of Epac hypertrophic signaling pathway.

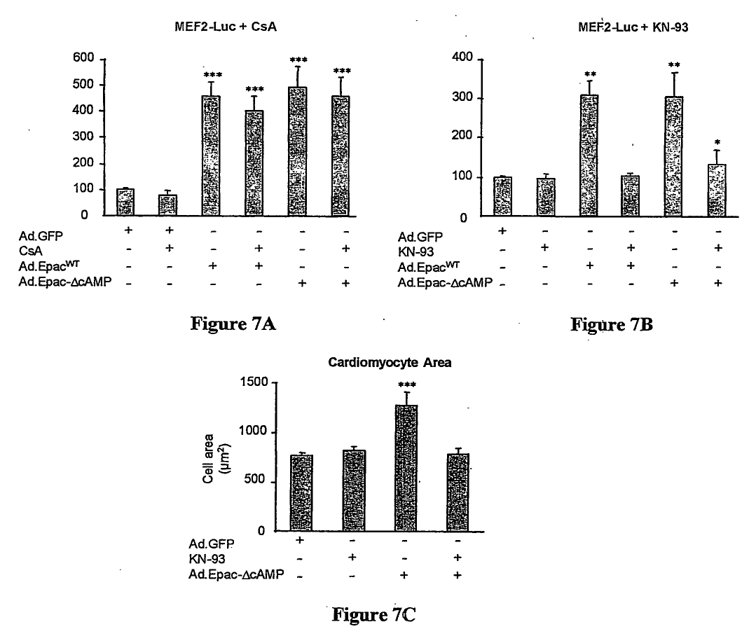

Next, the Inventors investigated whether MEF2 is also a target for Epac hypertrophic signaling. Infection of cardiomycytes with either Ad.EpacWT or Ad.Epac-DcAMP produced a strong increase in MEF2 transcriptional activity (FIG. 7A). Because calcineurin has been shown to stimulate MEF2 activity in cardiomyocytes (Zhang et al, 2002), the Inventors tested whether CsA could block the ability of Epac to enhance MEF2 transcriptional activity. As shown in FIG. 7A, CsA (0.5 μM) failed to inhibit Epac-induced MEF2 dependent luciferase activity indicating that calcineurin was not involved in this signaling pathway. In contrast, an inhibitor of CaMKII, KN-93 (1 μM) (Tombes et al, 1995) completely blocked the effect of EpacWT or Epac-DcAMP on MEF2-Luc activity (FIG. 7B). These results suggest a requirement for CaMKII in Epac-induced MEF2 transcriptional activity. Accordingly, the Inventors found that Ad.Epac-DcAMP-induced increase in cell surface area was significantly decreased by KN-93 (1 μM) (FIG. 7C).

### Involvement of Rac in Epac-Induced NFAT Dependent Cardiomyocyte Hypertrophy

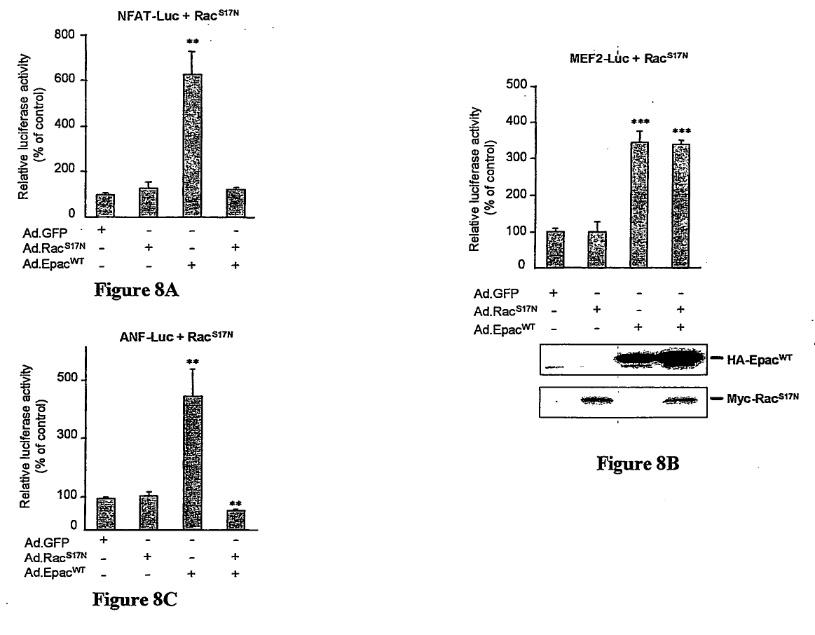

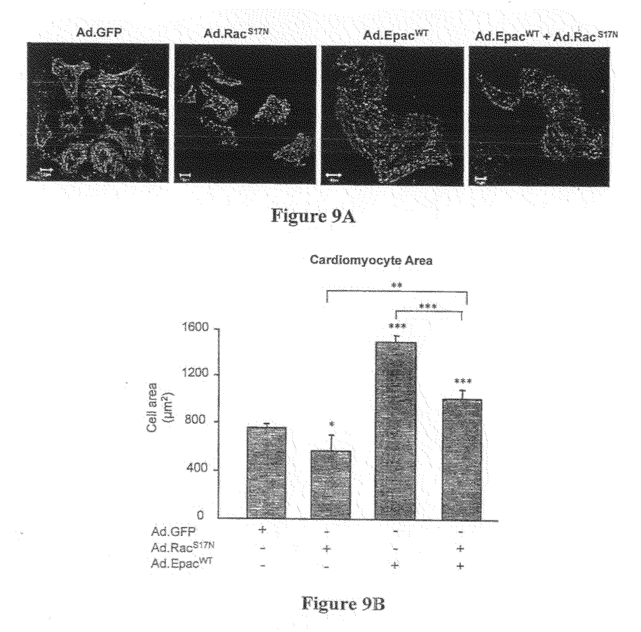

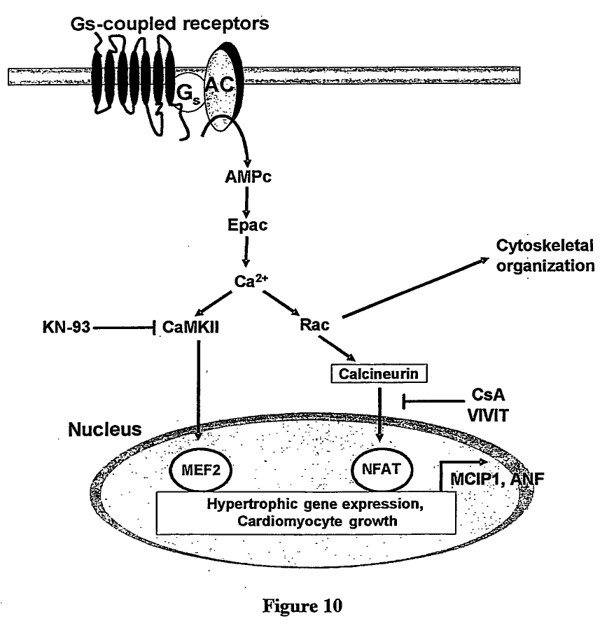

As Rac was found to be a downstream component of Epac signaling pathway (FIGS. 1A, 1B et 1C), the Inventors next examined the involvement of Rac in Epac-induced NFAT and MEF-2 transcriptional activities. Primary cardiomyocytes were transfected with either NFAT-Luc or MEF2-Luc, and the effect of RacS17N, a negative dominant form of Rac was tested on Epac-mediated activation of the Luc reporter constructs. Ad.RacS17N completely inhibited Epac-induced NFAT transcriptional activity (FIG. 8A) whereas Ad.RacS17N had no effect on MEF2 transcriptional activity in cells infected with either Ad.EpacWT (FIG. 8B) or Ad.Epac-DcAMP (data not shown). The involvement of Rac in Epac signaling pathway controlling cardiomyocyte hypertrophy was further supported by the observation that Ad.RacS17N inhibited Epac-induced ANF expression (FIG. 8C). Consistent with these findings, Ad.RacS17N inhibited Epac-induced cytoskeletal reorganization (FIG. 9A) and increase in cell surface areas (FIG. 9B). Altogether, these data clearly indicate that Epac activates calcineurin/NFAT signaling pathway via the small G protein, Rac (FIG. 10).

Epac1 is Increased in Patients with Heart Failure

To test whether the expression level of Epac is deregulated in heart failure, the Inventors performed the quantification of the major isoform of Epac in the heart, Epac1 by quantitative reverse transcriptase-polymerase chain reaction (RT-PCR). As shown in FIG. 12, Epac1 mRNA expression is significantly increased in patients with heart failure as compared to control samples.

### Discussion

The present study shows for the first time that cAMP-dependent activation of Epac induces cardiomyocyte hypertrophy in adult and neonatal rat cardiac myocytes. This is based on the observation that Epac activation leads to morphological changes, increases protein synthesis and induces alteration of gene expression of cardiac hypertrophic markers such as ANF and skeletal α-actin. In addition, the Inventors found that Epac activates a prohypertrophic signaling pathway which involves the Ca2+ sensitive phosphatase, calcineurin and its primary downstream effector, NFAT as well as MEF2. Epac-induced NFAT activation was dependent of Rac activity.

In their study, the Inventors showed that the Epac-specific cAMP analogue 8-CPT produces bursts of Ca2+ transients in neonatal myocytes. Low concentrations of 8-CPT (100 nM) elicited this effect in the presence of physiological external [Ca2+], while much higher ones (100 μM) were required in the absence of extracellular Ca2+. In other words, the apparent potency of 8-CPT to trigger intracellular Ca2+ spikes was enhanced in the presence of external Ca2+. Several explanations could be given to such an observation. A trivial one is that external Ca2+ directly facilitates 8-CPT penetration in the cell. Alternatively, these results suggest that 8-CPT acts through two distinct mechanisms, the activation of a Ca2+ influx with a high sensitivity and, with a lower sensitivity, the mobilisation of intracellular Ca2+.

In agreement with structural data (Enserink et al, 2002), the present results exclude the participation of HCN channels and PKA, the two other main cAMP targets in cardiac cells, as a mechanism of 8-CPT action on Ca2+ homeostasis. Indeed FIGS. 4B and 4D show that neither Cs+ nor the pharmacological PKA inhibitor H89 prevented 8-CPT-induced Ca2+ transients. Therefore, Epac-induced intracellular Ca2+ modulation might complement the previously reported ability of cAMP to enhance the activity of L-type Ca2+ channels and to sensitize RyR2 in a PKA-dependent manner in cardiac myocytes. The findings of the Inventors are in line with recent studies in pancreatic 1-cells and INS-1 insulin-secreting cells, demonstrating a PKA-independent mobilization of intracellular Ca2+ by the cAMP-elevating hormone glucagon-like peptide 1 and the implication of Epac in this effect (Tsuboi et al, 2003). In these cells, activation of endogenous Epac triggers Ca2+-induced Ca2+ release (Kang et al, 2003) and it is suggested that a functional coupling exists between Epac and the RyR in these cellular systems (Holz, 2004). Therefore, one could imagine in cardiac myoyctes, that Epac might interact with Ca2+ release channels or act via an intermediary to promote kinase-mediated phosphorylation of Ca2+ handling proteins. Interestingly, the small GTPase Rap1 which is an effector of Epac is suspected to play a role in cAMP-induced [Ca2+]i increase via SERCA3b in megakaryocytes (den Delder et al, 2002; Magnier et al, 1994). Alternatively, inositol 1,4,5-trisphosphate receptors (IP3Rs) might be involved in Epac-induced intracellular Ca2+ release since this cAMP-GEF has been previously shown to stimulate phospholipase C via the small GTPase Rap2B in HEK-293 cells and neuroblastoma cell lines (Keiper et al, 2004).

The Inventors found that the Epac-specific activator, 8-CPT induced Rac activation in primary cardiomyocytes and HL-1 cells. In addition, the Inventors showed that EpacWT or Epac-ΔcAMP expression also enhanced Rac activation in rat cardiomyocytes. This is in accordance with the recent findings of the Inventors showing that Epac induces Rac activation in a cAMP-dependent but PKA-independent manner in non-cardiac cells such as primary cortical neurons and CHO cells (Maillet et al, 2003). As the Inventors found that Rac was activated by Ca2+ following Epac stimulation, it is reasonable to think that Rac might be regulated by a GEF which is sensitive to Ca2+. Such a GEF has been reported for small GTPases of the Ras family (Keiper et al, 2004; Quilliam et al, 2002). Another molecular target which could be involved in Ca2+-dependent Rac activation is the Rho GDP-dissociation inhibitor (RhoGDI). Indeed, RhoGDI retains Rac into the cytoplasm and must dissociate to allow Rac to encounter its GEFs (Robbe et al, 2003; Schmidt & Hall, 2002). Recently, Price and colleagues (2003) have shown that Ca2+ induces a disruption of the Rac-Rho GDI complex leading to the translocation and activation of Rac in PC3 cells. Thus, one could speculate that such a mechanism might occur in cardiomyocytes and contribute to Epac-induced Ca2+-dependent Rac activation.

The Inventors report for the first time that Epac is implicated in the activation of NFAT and MEF2 in cardiac myocytes. The ability of Epac to stimulate NFAT activity was significantly inhibited by treatment with CsA and VIVIT, suggesting that calcineurin activity is regulated by Epac. Accordingly, the Inventors found that Epac up-regulates the expression of MCIP1, a well known modulator of calcineurin signaling which possesses a series of NFAT binding sites in its gene promoter (Vega et al, 2002; Yang et al, 2000). In addition, Ad.VIVIT partially reversed Epac-induced cardiomyocyte hypertrophy indicating that Epac is a new regulator of the hypertrophic calcineurin/NFAT signaling pathway. Interestingly, expression of a negative dominant form of Rac, RacS17N inhibited Epac-induced NFAT activation but failed to do so on MEF2 transcriptional activity. In accordance with the present results, RacS17N has been shown to block NFAT activation in immune cells (Jacinto et al, 1998).

A variety of Ca2+-dependent signal transduction pathways have been implicated in cardiac hypertrophy, but whether these pathways are dependent or interdependent is unclear (Molkentin, 2004; Zhang & Brown, 2004). Passier and colleagues (2000) have reported that calcineurin/NFAT and CaMKII/MEF2 pathways act in parallel and preferentially target different transcription factors to induce cardiac hypertrophy. However, in their study, the Inventors found that both calcineurin/NFAT and CaMKII/MEF2 pathways were required to trigger the hypertrophic program in neonatal cardiomyocytes as independent inhibition of one of these two signaling cascade was sufficient to block Epac-induced hypertrophic growth (FIGS. 7D, 8C, 9A). This raises the possibility that CaMKII and calcineurin pathways converge on common downstream target genes in the hypertrophic signaling pathway initiated by Epac. In line with this hypothesis, NFAT has been shown to bind DNA in conjunction with MEF2, in promoter/enhancer regions controlling transcription of genes encoding proteins of the slow-fiber program (Chin et al, 1998). In addition, two hypertrophic signaling modules, calcineurin/NFAT and MEK1-ERK1/2 coordinately regulate cardiac growth through two distinct mechanisms (Sanna et al., 2005).

Thus, the Inventors propose a new cAMP signaling pathway in which a prolonged activation of Epac leads to a sustained increase in [Ca2+]i which then activates CaMKII and Rac. The latter increases calcineurin/NFAT activation. This signaling cascade activates hypertrophic gene expression and induces the morphological aspects of cardiac myocyte hypertrophy (FIG. 10). These results thus open new insights into the signaling pathways by which cAMP may mediate its biological effects in cardiomyocytes and thus, raise the question of the identity of the neurohormonal factors which are involved in the regulation of Epac activity in cardiac myocytes. Finally, the fact that the Inventors found an upregulation of Epac1 gene expression in patients with heart failure suggests that this guanine nucleotide exchange factor contribute to the development of this pathology and strengthens the hypothesis that inhibitors of Epac are useful for the treatment of such cardiac pathology.

### Example 2

### Construction of siRNAs that Specifically Inhibit the Expression of Epac

To provide siRNAs that specifically inhibit the expression of Epac, the following guidelines are used according to Elbashir et al., 2002: 1) Selection of the target region from the open reading frame (ORF) of the cDNA sequence preferably 50 to 100 nucleotides downstream of the start codon. 2) Determination of a 21 nucleotide sequence in the target mRNA that begins with an AA dinucleotide (Elbashir et al., 2001). Thus sequences are 5′-AA(N19)UU, where N is any nucleotide. Sequences must contain approximately 50% G/C. 3) Blast-search (www.ncbi.nih.go/BLAST) the selected siRNA sequences against EST libraries or mRNA sequences of the respective organism to ensure that only a single gene is targeted. Any target sequences with more than 16 to 17 contiguous base pairs of homology to other coding sequences must be eliminated. 4) Synthesis of several siRNA sequences are advisable to control for the specificity of the knock-down experiments.

Then A) the software of Qiagen company or the software “online target finder” of the custom chemical synthesis service for siRNA present on the website of the RNA company Ambion is used to design potential sequences based on the guidelines described above.

B) Selected siRNA sequences are purchased fully deprotected, desalted with PAGE purification and delivered in dry form along with RNAse-free water and 5× annealing buffer.

C) The next step is the annealing of the siRNAs to produce siRNA duplexes ready to use for RNAi transfection experiments. Annealing can be performed as follows: incubation of equimolecular concentration of oligonucleotides (20 μM) in annealing buffer for 1 min at 90° C., centrifugation (15 s) and incubation at 37° C. for 1 h. The siRNA duplex (20 μM) is ready to use for RNAi experiments and can be stored at −20° C. and undergo multiple freeze-thaw cycles.

D) Transfection of siRNA duplex is performed using the Lipofectamine 2000 (Clontech) in primary neonatal rat cardiomyocytes with optimum medium according to the manufacturer's instructions and assay for silencing is carried out 1 and 2 days after transfection. The rat neonatal cardiomyocytes are seeded the previous day in 12 well plates at 40 to 50% of confluence. Transfection efficiency is typically around 90-95%, although it is cell type dependent.

E) The effect of silencing of the several isoforms of Epac (Epac1 accession number NM_{—}006105 of sequence SEQ ID NO: 1, Epac2 NM_{—}007023 of sequence SEQ ID NO: 3 and Repac BC_{—}039203 of sequence SEQ ID NO: 5) is investigated on various parameters of cardiac hypertrophy (cell size, hypertrophic gene markers) upon cardiomyocytes treatment with various hypertrophic stimuli (i.e: isoproterenol, angiotensin II, phenylephrine, endotheline-1), in the aim of future applications in human cardio-physiopathology.

### Example 3

### Non Human Model of Cardiac Hypertrophy Induced by Epac

Cardiac hypertrophy has been induced in mouse by the specific expression of a positive dominant form of Epac1 (Epac-ΔcAMP) in mouse cardiomyocytes. The cDNA coding for the human form of Epac1 lacking its first 322 amino acids (EpacΔcAMP) and containing a HA epitope in its N terminus (de Rooij et al., Nature 396: 474-477) was fused to the α-Myosin Heavy Chain cardiac specific promoter (Gulick et al., 1991) (FIG. 14). The plasmid construct containing the α-Myosin Heavy Chain promoter cloned upstream HA-Epac-ΔcAMP (FIG. 14) is then linearized and inserted into the Hypoxanthine PhosphoRibosylTransferase (hprt) locus of BPES cells hprt negative, according to the technique described by Farhadi et al. 2003 and to the international application WO2005/005619. Positive clones are then microinjected into mouse blastocytes (C57Bl/6 genetic background), which are grafted into foster females. Offspring is screened for the presence of the transgene by Southern blotting analysis performed on DNA extracted from tail biopsies. Second generation of transgenic heterozygous animals is sacrificed to check for the RNA and the protein expressions of the transgene and its functional activity in the heart.

### REFERENCES

Aramburu J, Yaffe M B, Lopez-Rodriguez C, Cantley L C, Hogan P G, Rao A (1999) Affinity-driven peptide selection of an NFAT inhibitor more selective than cyclosporin A. Science 285: 2129-2133
Avruch J, Khokhlatchev A, Kyriakis J M, Luo Z, Tzivion G, Vavvas D, Zhang X F. Ras activation of the Raf kinase: tyrosine kinase recruitment of the MAP kinase cascade. Recent Prog Horm Res. 2001; 56:127-155.
Balke C W, Shorofsky S R (1998) Alterations in calcium handling in cardiac hypertrophy and heart failure. Cardiovasc Res 37: 290-299
Bers D M, Ziolo M T (2001) When is cAMP not cAMP? Effects of compartmentalization. Circ Res 89: 373-375
Bos J L (2003) Epac: a new cAMP target and new avenues in cAMP research. Nat Rev Mol Cell Biol 2: 369-377
Chien K R, Knowlton K U, Zhu H, Chien S (1991) Regulation of cardiac gene expression during myocardial growth and hypertrophy: molecular studies of an adaptive physiologic response. FASEB J 5: 3037-3046
Chin E R, Olson E N, Richardson J A, Yang Q, Humphries C, Shelton J M, Wu H, Zhu W, Bassel-Duby R, Williams R S (1998) A calcineurin-dependent transcriptional pathway controls skeletal muscle fiber type. Genes Dev 12: 2499-2509
Christensen A E, Selheim F, de Rooij J, Dremier S, Schwede F, Dao K K, Martinez A, Maenhaut C, Bos J L, Genieser H G, Doskeland S O (2003) cAMP analog mapping of Epac1 and cAMP kinase. Discriminating analogs demonstrate that Epac and cAMP kinase act synergistically to promote PC-12 cell neurite extension. J Biol Chem 278: 35394-35402
Clerk A, Pham F H, Fuller S J, Sahai E, Aktories K, Marais R, Marshall C, Sugden P H (2001) Regulation of mitogen-activated protein kinases in cardiac myocytes through the small G protein Rac1. Mol Cell Biol 21: 1173-1184
Clerk A, Sugden P H (2000) Small guanine nucleotide-binding proteins and myocardial hypertrophy. Circ Res 86: 1019-1023
de Rooij J, Rehmann H, van Triest M, Cool R H, Wittinghofer A, Bos J L (2000) Mechanism of regulation of the Epac family of cAMP-dependent RapGEFs. J Biol Chem 275: 20829-20836
de Rooij J, Zwartkruis F J T, Verheijen M H G, Cool R H, Nijman S M B, Wittinghofers A, Bos J L (1998) Epac is a Rap1 guanine-nucleotide-exchange factor directly activated by cyclic AMP. Nature 396: 474-477
den Dekker E, Heemskerk J W, Gorter G, van der Vuurst H, Donath J, Kroner C, Mikoshiba K, Akkerman J W (2002) Cyclic AMP raises intracellular Ca2+ in human megakaryocytes independent of protein kinase A. Arterioscler Thromb Vasc Bio 22: 179-186
Elbashir S M, Harborth J, Weber K, Tuschl T. (2002) Analysis of gene function in somatic mammalian cells using small interfering RNAs Methods 26: 199-213.
Elbashir S M, Martinez J, Patkaniowska A, Lendeckel W, Tuschl T. (2001) Functional anatomy of siRNA for mediating efficient RNAi in drosophyla melanogaster embryo lysat EMBO J 20: 6877-6888.
Enserink J M, Christensen A E, de Rooij J, van Triest M, Schwede F, Genieser H G, Doskeland S O, Blank J L, Bos J L (2002) A novel Epac-specific cAMP analogue demonstrates independent regulation of Rap1 and ERK. Nat Cell Biol 4: 901-906
Enserink J M, Price L S, Methi T, Mahic M, Sonnenberg A, Bos J L, Tasken K (2004) The cAMP-Epac-Rap1 pathway regulates cell spreading and cell adhesion to laminin-5 through the alpha3beta1 integrin but not the alpha6beta4 integrin. J Biol Chem 279: 44889-44896
Er F, Larbig R, Ludwig A, Biel M, Hofmann F, Beuckelmann D J, Hoppe U C (2003) Dominant-negative suppression of HCN channels markedly reduces the native pacemaker current I(f) and undermines spontaneous beating of neonatal cardiomyocytes. Circulation 107: 485-489
Farhadi H F, Lepage P, Forghani R, Friedman H C H., Orfali W, Jasmin L, Miller W, Hudson T J, Peterson A C. (2003) A combinatorial Network of Evolutionarily Conserved Myelin Basic Protein Regulatory Sequences Confers Distinct Glial-Specific Phenotypes. J. of Neurosci. 23: 10214-10223
Fimia G M, Sassone-Corsi P (2001) Cyclic AMP signaling. J Cell Sci 114: 1971-1972
Frey N, McKinsey T A, Olson E N (2000) Decoding calcium signals involved in cardiac growth and function. Nat Med 6: 1221-1227
Gulick J, Subramaniam A, Neumann J, Robbins J. (1991) Isolation and characterization of the mouse cardiac myosin heavy chain genes. J. Biol. Chem. 266: 9180-9185.
Hall A. (1998) Rho GTPases and the actin cytoskeleton. Science 279: 509-514
Hennersdorf M G, Strauer B E. Arterial hypertension and cardiac arrhythmias. J. Hypertens. 2001; 19:167-177
Hochbaum D, Tanos T, Ribeiro-Neto F, Altschuler D, Coso O A. (2003) Activation of JNK by Epac is independent of its activity as a Rap guanine nucleotide exchanger. J. Biol. Chem. 278: 33738-33746.
Holz G G (2004) Epac: A new cAMP-binding protein in support of glucagon-like peptide-1 receptor-mediated signal transduction in the pancreatic beta-cell. Diabetes 53: 5-13
Ichiba T, Hoshi Y, Eto Y, Tajima N, Kuraishi Y. (1999) Characterization of GFR, a novel guanine nucleotide exchange factor for Rap1. FEBS Lett. 457: 85-89
Jacinto E, Werlen G, Karin M (1998) Cooperation between Syk and Rac1 leads to synergistic JNK activation in T lymphocytes. Immunity 8: 31-41
Kang G, Joseph J W, Chepurny O G, Monaco M, Wheeler M B, Bos J L, Schwede F, Genieser H G, Holz G G (2003) Epac-selective cAMP Analog 8-pCPT-2′-O-Me-cAMP as a stimulus for Ca2+-induced Ca2+ release and exocytosis in pancreatic beta cells. J Biol Chem 278: 8279-8285
Kawasaki H, Springett G M, Mochizuki N, Toki S, Nakaya M, Matsuda M, Housman D E, Graybel A M (1998) A family of cAMP-binding that directly activate Rap1. Science 282: 2275-2279
Keiper M, Stope M B, Szatkowski D, Bohm A, Tysack K, Vom Dorp F, Saur O, Oude Weernink P A, Evellin S, Jakobs K H, Schmidt M (2004) Epac- and Ca2+-controlled activation, of Ras and extracellular signal-regulated kinases by Gs-coupled receptors. J Biol Chem 279: 46497-46508
Liang Q, Bueno O F, Wilkins B J, Kuan C Y, Xia Y, Molkentin J D (2003) c-Jun N-terminal kinases (JNK) antagonize cardiac growth through cross-talk with calcineurin-NFAT signaling. EMBO J 22:5079-5089
Luttrell L M. Activation and targeting of mitogen-activated protein kinases by G-protein-coupled receptors. Can J Physiol Pharmacol. 2002; 80:375-382
Magnier C, Bredoux R, Kovacs T, Quarck R, Papp B, Corvazier E, de Gunzburg J, Enouf J (1994) Correlated expression of the 97 kDa sarcoendoplasmic reticulum Ca2+-ATPase and Rap1B in platelets and various cell lines. Biochem J 297: 343-350
Maillet M, Robert S J, Cacquevel M, Gastineau M, Vivien D, Bertoglio J, Zugaza J L, Fishmeister R, Lezoualc'h F (2003) Crosstalk between Rap1 and Rac regulates secretion of sAP\tilde{P}. Nature Cell Biol 5: 633-639
McKinsey T A, Olson E N (2004). Cardiac histone acetylation-therapeutic opportunities abound. Trends Genet 20: 206-213
Molkentin J D (2004) Calcineurin-NFAT signaling regulates the cardiac hypertrophic response in coordination with the MAPKs. Cardiovasc Res 63: 467-475
Moodie S A, Wolfman A. The 3Rs of life: Ras, Raf and growth regulation. Trends Genet. 1994; 10:44-48
Morel E, Marcantoni A, Gastineau M, Birkledal R, Garnier A, Lompré A M, Vandecasteele G, Lezoualc'h F. (2005) The cAMP-binding protein Epac induces cardiomyocyte hypertrophy. Circ. Res. 97: 1296-1304.
Morrison D K, Cutler R E. The complexity of Raf-1 regulation. Curr Opin Cell Biol. 1997; 9:174-179
Ozaki N, Shibasaki T, Kashima Y, Miki T, Takahashi K, Ueno H, Sunaga Y, Yano H, Matsuura Y, Iwanaga T, Takai Y, Seino S (2000) cAMP-GEFII is a direct target of cAMP in regulated exocytosis. Nat Cell Biol 2: 805-811
Passier R, Zeng H, Frey N, Naya F J, Nicol R L, McKinsey T A, Overbeek P, Richardson J A, Grant S R, Olson E N (2000) CaM kinase signaling induces cardiac hypertrophy and activates the MEF2 transcription factor in vivo. J Clin Invest 105: 1395-1406
Pracyk J. B., Tanaka K., Hegland D. D., Kim K. S., Sethi R., Rovira R., Blazina D. R., Lee L., Bruder J. T., Kovesdi I., Goldshmidt-Clermont P. J., Irani K., Finkel T (1998) A requirement for the rac1 GTPase in the signal transduction pathway leading to cardiac myocyte hypertrophy. J Clin Invest 102: 929-937
Price L S, Langeslag M, Klooster J P T, Hordijk P L, Jalink K, Collard J G (2003) Calcium signaling regulates translocation and activation of Rac. J Biol Chem 278: 39413-39421
Quilliam L A, Rebhun J F, Castro A F (2002) A growing family of guanine nucleotide exchange factors is responsible for activation of Ras-family GTPases. Prog Nucleic Acid Res Mol Biol 71: 391-444
Rangarajan S, Enserink J M, Kuiperij H B, de Rooij J, Price L S, Schwede F, Bos J L (2003) Cyclic AMP induces integrin-mediated cell adhesion through Epac and Rap1 upon stimulation of the beta 2-adrenergic receptor. J Cell Biol 160: 487-493
Rehmann H, Schwede F, Doskeland S O, Wittinghofer A, Bos J L (2003) Ligand-mediated activation of the cAMP-responsive guanine nucleotide exchange factor Epac. J Biol Chem 278: 38548-38556
Robbe K, Otto-Bruc A, Chardin P, Antonny B (2003) Dissociation of GDP dissociation inhibitor and membrane translocation are required for efficient activation of Rac by the Dbl homology-pleckstrin homology region of Tiam. J Biol Chem 278: 4756-4762
Sadoshima J, Izumo S. The cellular and molecular response of cardiac myocytes to mechanical stress. Annu Rev Physiol. 1997; 59:551-571
Sadoshima J, Xu Y, Slayter H S, Izumo S. Autocrine release of angiotensin II mediates stretch-induced hypertrophy of cardiac myocytes in vitro. Cell. 1993; 75:977-984
Sanna B, Bueno O F, Dai Y S, Wilkins B J, Molkentin J D (2005) Direct and indirect interactions between calcineurin-NFAT and MEK1-extracellular signal-regulated kinase 1/2 signaling pathways regulate cardiac gene expression and cellular growth. Mol Cell Biol 25: 865-878
Schmidt A, Hall A. (2002) Guanine nucleotide exchange factors for Rho GTPases: turning on the switch. Genes Dev 16: 1587-1609
Sussman M A, Welch S, Walker A, Klevitsky R, Hewett T E, Price R L, Schaefer E, Yager K (2000) Altered focal adhesion regulation correlates with cardiomyopathy in mice expressing constitutively active Rac1. J Clin Invest 105: 875-886
Tombes R M, Grant S, Westin E H, Krystal G (1995) G1 cell cycle arrest and apoptosis are induced in NIH 3T3 cells by KN-93, an inhibitor of CaMK-II (the multifunctional Ca2+/CaM kinase). Cell Growth Differ 6: 1063-1070
Tsuboi T, da Silva Xavier G, Holz G G, Jouaville L S, Thomas A P, Rutter G A. (2003) Glucagon-like peptide-1 mobilizes intracellular Ca2+ and stimulates mitochondrial ATP synthesis in pancreatic MIN6 beta-cells. Biochem J 369: 287-299
Vakili B A, Okin P M, Devereux R B. Prognostic implications of left ventricular hypertrophy. Am Heart J. 2001; 141:334-341
van den Berghe N, Cool R H, Horn G, Wittinghofer A. Biochemical characterization of C3G: an exchange factor that discriminates between Rap1 and Rap2 and is not inhibited by Rap1A(S17N). (1997) Oncogene 15: 845-850
Vega R B, Yang J, Rothermel B A, Bassel-Duby R, Williams R S (2002) Multiple domains of MCIP1 contribute to inhibition of calcineurin activity. J Biol Chem 277: 30401-30407
Verde, I., Vandecasteele, G., Lezoualc'h, F., and Fischmeister, R. (1999) Characterization of the cyclic nucleotide phosphodiesterase subtypes involved in the regulation of the L-type Ca2+ current in rat ventricular myocytes. Br. J. Pharmacol. 127: 65-74.
Wollert K C, Taga T, Saito M, Narazaki M, Kishimoto T, Glembotski C C, Vernallis A B, Heath J K, Pennica D, Wood W I, Chien K R (1996) Cardiotrophin-1 activates a distinct form of cardiac muscle cell hypertrophy. Assembly of sarcomeric units in series VIA gp130/leukemia inhibitory factor receptor-dependent pathways. J Biol Chem 271: 9535-9545
Yang J, Rothermel B, Vega R B, Frey N, McKinsey T A, Olson E N, Bassel-Duby R, Williams R S (2000) Independent Signals control expression of the calcineurin inhibitory proteins MCIP1 and MCIP2 in striated muscles. Circ Res 87: 61-68
Zhang C L, McKinsey T A, Chang S, Antos C L, Hill J A, Olson E N (2002) Class II histone deacetylases act as signal-responsive repressors of cardiac hypertrophy. Cell 110: 479-488
Zhang T, Brown J H (2004) Role of Ca2+/calmodulin-dependent protein kinase II in cardiac hypertrophy and heart failure. Cardiovasc Res 63: 476-486

1. The use of at least one Epac (Exchange Protein directly Activated by cAMP) antagonist for the manufacture of a medicament intended for the prevention or the treatment of pathologies selected from the list comprising cardiac hypertrophy, cardiac arrhythmias, valvulopathies, diastolic dysfunction, chronic heart failure, ischemic heart failure, and myocarditis.

2. The use according to claim 1, wherein the antagonist is selected from the list comprising Epac activation inhibitors, Epac activity inhibitors, Epac intracellular localization disruption agents, and Epac expression inhibitors.

3. The use according to claim 1, wherein the antagonist is an Epac activation inhibitor selected from the list comprising:
antibodies, fragments thereof, or aptamers, directed against Epac cAMP binding sites,
cAMP analogues, such as cAMP derivatives or 8-(4-chlorophenylthio)-2′-O-methyladenosine-3′-5-cyclic monophosphate (8-CPT-2′-O-Me-cAMP) derivatives,
Brefeldin A or derivatives thereof.

4. The use according to claim 1, wherein the antagonist is an Epac activity inhibitor, in particular an inhibitor of the Guanine nucleotide Exchange Factor (GEF) domain of Epac, or an inhibitor of the Ras Exchange Motif (REM) domain of Epac, selected from the list comprising:
antibodies, fragments thereof, or aptamers, directed against the GEF domain or the REM domain of Epac,
Brefeldin A or derivatives thereof.

5. The use according to claim 1, wherein the antagonist is an Epac intracellular localization disruption agent, selected from the list comprising:
antibodies, fragments thereof, or aptamers, directed against an Epac cellular localization domain, such as the Dishevelled EgI-10 Pleckstrin (DEP) domain,
Brefeldin A or derivatives thereof.

6. The use according to claim 1, wherein the antagonist is an Epac expression inhibitor selected from the list comprising:
an antisense nucleic acid directed against Epac mRNAs,
a single stranded DNA, directed against Epac double strand DNA,
a double stranded RNA, a siRNA or a sbJRNA, comprising Epac nucleic acid sequences,
a ribozyme directed against Epac mRNAs.

7. A method for treating a pathology selected from the list comprising cardiac hypertrophy, cardiac arrhythmias, valvulopathies, diastolic dysfunction, chronic heart failure, ischemic heart failure, and myocarditis, in a patient, comprising administering to said patient a therapeutically effective amount of at least one Epac antagonist.

8. The method according to claim 7, wherein the antagonist is selected from the list comprising Epac activation inhibitors, Epac activity inhibitors, Epac intracellular localization disruption agents, and Epac expression inhibitors.

9. The method according to claim 7, wherein the antagonist is an Epac activation inhibitor selected from the list comprising:
antibodies, fragments thereof, or aptamers, directed against Epac cAMP binding sites,
cAMP analogues, such as cAMP derivatives or 8-(4-chlorophenylthio)-2′-O-methyladenosine-3′-5-cyclic monophosphate (8-CPT-2′-O-Me-cAMP) derivatives,
Brefeldin A or derivatives thereof.

10. The method according to claim 7, wherein the antagonist is an Epac activity inhibitor, in particular an inhibitor of the Guanine nucleotide Exchange Factor (GEF) domain of Epac, or an inhibitor of the Ras Exchange Motif (REM) domain of Epac, selected from the list comprising:
antibodies, fragments thereof, or aptamers, directed against the GEF domain or the REM domain of Epac,
Brefeldin A or derivatives thereof.

11. The method according to claim 7, wherein the antagonist is an Epac intracellular localization disruption agent, selected from the list comprising:
antibodies, fragments thereof, or aptamers, directed against an Epac cellular localization domain, such as the Dishevelled EgI-10 Pleckstrin (DEP) domain,
Brefeldin A or derivatives thereof.

12. The method according to claim 7, wherein the antagonist is an Epac expression inhibitor selected from the list comprising:
an antisense nucleic acid directed against Epac mRNAs,
a double stranded RNA, a siRNA or a shRNA comprising Epac nucleic acid sequences,
a ribozyme directed against Epac mRNAs.

13. Pharmaceutical compositions comprising as active substance an Epac expression inhibitor selected from the list comprising:
an antisense nucleic acid directed against Epac mRNAs,
a double stranded RNA, a siRNA or a shRNA comprising Epac nucleic acid sequences,
a ribozyme directed against Epac mRNAs. in association with a pharmaceutically acceptable carrier.

14. Pharmaceutical compositions comprising as active substance an Epac activation inhibitor, an Epac activity inhibitor, or an Epac intracellular localization disruption agent, selected from the list comprising:
antibodies, fragments thereof, or aptamers, directed against Epac cAMP binding sites,
antibodies, fragments thereof, or aptamers, directed against the GEF domain or the REM domain of Epac,
antibodies, fragments thereof, or aptamers, directed against an Epac cellular localization domain, such as the DEP domain, in association with a pharmaceutically acceptable carrier.

15. The use of Epac for screening compounds intended for the prevention or the treatment of pathologies selected from the list comprising cardiac hypertrophy, cardiac arrhythmias, valvulopathies, diastolic dysfunction, chronic heart failure, ischemic heart failure, and myocarditis.

16. A method for screening compounds intended for the prevention or the treatment of pathologies selected from the list comprising cardiac hypertrophy, cardiac arrhythmias, valvulopathies, diastolic dysfunction, chronic heart failure, ischemic heart failure, and myocarditis, comprising the steps of:
contacting Epac with a compound to screen in the presence of cAMP or an Epac activating c AMP analogue,
assaying Epac activation,
selecting compounds which inhibit Epac activation, in particular compounds which inhibit at least 30% of Epac activation, as compared to Epac activation in the absence of said compounds.

17. The method according to claim 16, wherein Epac is also contacted with an effector protein liable to be activated upon Epac activation, such as Rap1, Rap2, Rac or Ras, and Epac activation is assessed by measuring the activity of said effector protein.

18. A method for screening compounds intended for the prevention or the treatment of pathologies selected from the list comprising cardiac hypertrophy, cardiac arrhythmias, valvulopathies, diastolic dysfunction, chronic heart failure, ischemic heart failure, and myocarditis comprising the steps of:
contacting cardiomyocytes having increased Epac activity as compared to normal cardiomyocytes with compounds to screen,
assessing the hypertrophic properties of said cardiomyocytes having increased Epac activity,
selecting compounds which inhibit the hypertrophic properties of said cardiomyocytes having increased Epac activity, as compared to the hypertrophic properties of cardiomyocytes having increased Epac activity not treated by said compounds.

19. The method according to claim 16, wherein the compounds to screen are:
cAMP analogues, such as cAMP derivatives or 8-(4-chlorophenylthio)-2′-O-methyladenosine-3′-5-cyclic monophosphate (8-CPT-2′-O-Me-cAMP) derivatives, or
Brefeldin A derivatives.

20. A non-human transgenic mammal for use as a model of cardiac hypertrophy, wherein Epac activity is increased with respect the corresponding wild type non-human mammal.

21. A non-human transgenic mammal for use as a model of cardiac hypertrophy according to claim 20, wherein Epac is over-expressed with respect the corresponding wild type non-human mammal, in particular said non-human transgenic mammal comprises Epac coding sequences under the control of cardiac-specific promoters having a stronger transcription activity than Epac natural promoter, such as the promoter of the 6-Myosin Heavy Chain.

22. A non-human transgenic mammal for use as a model of cardiac hypertrophy according to claim 20, wherein said non-human transgenic mammal comprises nucleic sequences encoding a constitutively activated form of Epac lacking the activating cAMP binding domain.

23. A non-human transgenic mammal for use as a model of cardiac hypertrophy according to claim 20, wherein said mammal is a mouse.


SEQUENCE LISTING (18 sequences)

SEQ ID NO: 1 - DNA, 3327 bp, Homo sapiens
  CDS (343)..(2985)
gcccccacccctctactcacccaagaggagcctctccaccgagccctgcatcctgctgccctggcagccagtccagctaggcactggcccagttcactgtacctggacttacccactggcaccctccggatccccttatcaaagctgatgggggtcgctggggaccccccagccttttgctagagcctgcacggctgtgcgagcttgaaaagaaacatgaaggtgggctggccaggtgagagctgctggcaggtgggcctggctgtggaggatagcccagctctgggagcaccgcgggtgggagccctccctgacgtggtgccggaggggacactactcaacatggtgttgagaaggatgcaccggccccgaagctgctcctaccagctgctgctggagcaccagcgtccgagctgcatccaggggctgcgctggacaccactcaccaacagcgaggagtccctggatttcagcgagagcctggagcaggcctccacagagcgggtgctcagggctgggaggcagctgcatcggcatctgctggccacctgcccaaacctcatccgagaccggaagtaccaccttaggctctatcggcagtgctgctctggccgggagctggtggatgggatcttggccctgggacttggggtccattcccggagccaagttgtgggaatctgccaggtgctgctggatgaaggtgccctctgccatgtgaaacacgactgggccttccaggaccgagatgcccaattctaccggttccccgggcccgagcccgagcccgtgggaactcatgagatggaggaggagttggccgaagctgtggccctgctctcccagcgggggcctgacgccctgctcactgtggcacttcgaaagcccccaggtcagcgcacggatgaagagctggacctcatctttgaggagctgctgcacatcaaggctgtggcccacctctccaactcggtgaagcgagaattagcggctgttctgctctttgaaccacacagcaaggcagggaccgtgttgttcagccagggggacaagggcacttcgtggtacattatctggaagggatctgtcaacgtggtgacccatggcaaggggctggtgaccaccctgcatgagggagatgattttggacagctggctctggtgaatgatgcaccccgggcagccaccatcatcctgcgagaagacaactgtcatttcctgcgtgtggacaagcaggacttcaaccgtatcatcaaggatgtggaggcaaagaccatgcggctggaagaacatggcaaagtggtgctggtgctggagagagcctctcagggcgccggcccttcccgacccccaaccccaggcaggaaccggtatacagtgatgtctggcaccccagagaagatcctagagcttctgttggaggccatgggaccagattccagtgctcatgacccaacagagacattcctcagcgacttcctcctgacccacagggtcttcatgcccagcgcccaactctgcgctgcccttctgcaccacttccatgtggagcctgcgggtggcagcgagcaggagcgcagcacctacgtctgcaacaagaggcagcagatcttgcggctggtcagccagtgggtggccctgtatggctccatgctccacactgaccctgtggccaccagcttcctccagaaactctcagacctggtgggcagggacacccgactcagcaacctgctgagggagcagtggccagagaggcggcgatgccacaggttggagaatggctgtgggaatgcatctcctcagatgaaggcccggaacttgcctgtttggctccccaaccaggacgagccccttcctggcagcagctgtgccatccaagttggggataaagtcccctatgacatctgccggccagaccactcagtgttgaccctgcagctgcctgtgacagcctccgtgagagaggtgatggcagcgttggcccaggaggatggctggaccaaggggcaggtgctggtgaaggtcaattctgcaggtgatgccattggcctgcagccagatgcccgtggtgtggccacatctctggggctcaatgagcgtctctttgttgtcaacccacaggaagtgcatgagctgatcccacaccctgaccagctggggcccactgtgggctctgctgaggggctggacctggtgagtgccaaggacctggcaggccagctgacggaccacgactggagcctcttcaacagtatccaccaggtggagctgatccactatgtgctgggcccccagcatctgcgggatgtcaccaccgccaacctggagcgcttcatgcgccgcttcaatgagctgcagtactgggtggccaccgagctgtgtctctgccccgtgcccggcccccgggcccagctgctcaggaagttcattaagctggcggcccacctcaaggagcagaagaatctcaattccttctttgccgtcatgtttggcctcagcaactcggccatcagccgcctagcccacacctgggagcggctgcctcacaaagtccggaagctgtactccgccctcgagaggctgctggatccctcatggaaccaccgggtataccgactggccctcgccaagctctcccctcctgtcatccccttcatgccccttcttctcaaagacatgaccttcattcatgagggaaaccacacactagtggagaatctcatcaactttgagaagatgagaatgatggccagagccgcgcggatgctgcaccactgccgaagccacaaccctgtgcctctctcaccactcagaagccgagtttcccacctccacgaggacagccaggtggcgaggatttccacatgctcggagcagtccctgagcacccggagtccagccagcacctgggcttatgtccagcagctgaaggtcattgacaaccagcgggaactctcccgcctctcccgagagctggagccatgaggaggggctgggactggagctggagcaggcacttgcagccgggaaagccagggtgtgccgggccaagatactcacaggctggccacagctgggcaaggctctccgtggagtggactcgagtccctggagcaggcagtgtggaggcagccatcccctgtgatgactggcagctaaggaggacctcggagtggacccgagccaggaataacgaatgacccaaggccaaggaagggaggacagagaggccccaggagtgggtggagagtggagtgcgctgggacgttgtgtgcaatagagaggtctccacaccagatgtcttccagaaaaaaaaaaaaaaaa

SEQ ID NO: 2 - protein, 881 aa, Homo sapiens
MVLRRMHRPRSCSYQLLLEHQRPSCIQGLRWTPLTNSEESLDFSESLEQASTERVLRAGRQLHRHLLATCPNLIRDRKYHLRLYRQCCSGRELVDGILALGLGVHSRSQVVGICQVLLDEGALCHVKHDWAFQDRDAQFYRFPGPEPEPVGTHEMEEELAEAVALLSQRGPDALLTVALRKPPGQRTDEELDLIFEELLHIKAVAHLSNSVKRELAAVLLFEPHSKAGTVLFSQGDKGTSWYIIWKGSVNVVTHGKGLVTTLHEGDDFGQLALVNDAPRAATIILREDNCHFLRVDKQDFNRIIKDVEAKTMRLEEHGKVVLVLERASQGAGPSRPPTPGRNRYTVMSGTPEKILELLLEAMGPDSSAHDPTETFLSDFLLTHRVFMPSAQLCAALLHHFHVEPAGGSEQERSTYVCNKRQQILRLVSQWVALYGSMLHTDPVATSFLQKLSDLVGRDTRLSNLLREQWPERRRCHRLENGCGNASPQMKARNLPVWLPNQDEPLPGSSCAIQVGDKVPYDICRPDHSVLTLQLPVTASVREVMAALAQEDGWTKGQVLVKVNSAGDAIGLQPDARGVATSLGLNERLFVVNPQEVHELIPHPDQLGPTVGSAEGLDLVSAKDLAGQLTDHDWSLFNSIHQVELIHYVLGPQHLRDVTTANLERFMRRFNELQYWVATELCLCPVPGPRAQLLRKFIKLAAHLKEQKNLNSFFAVMFGLSNSAISRLAHTWERLPHKVRKLYSALERLLDPSWNHRVYRLALAKLSPPVIPFMPLLLKDMTFIHEGNHTLVENLINFEKMRMMARAARMLHHCRSHNPVPLSPLRSRVSHLHEDSQVARISTCSEQSLSTRSPASTWAYVQQLKVIDNQRELSRLSRELEP

SEQ ID NO: 3 - DNA, 4278 bp, Homo sapiens
  CDS (109)..(3141)
cgggaggagcggggtccgcgcggcggacgaggcgggggcaggaggcgcaggcagagcgagcgcgggaggtcgccgcagcacagaggacaccgcgcgccgccgctcaacatggtcgctgcgcacgctgcccattcttcctcctctgccgagtggatcgcctgcctggataaaagaccactggagcgatccagcgaagatgtggatataatcttcactcgactgaaagaagttaaagcttttgagaaatttcacccaaatctccttcatcagatttgcttatgtggttattatgagaatctggaaaagggaataacattatttcgccagggtgatattggaacaaactggtatgctgtcctggcagggtctttggatgttaaagtatctgagaccagcagtcaccaggatgctgtgaccatctgtaccctgggaattgggacggcctttggagagtccattctggacaacacaccccgccatgcaaccatcgttaccagggagagcagtgaactgctccgcatcgagcagaaggacttcaaggcactatgggagaaatatcgacagtatatggcaggacttctggctcctccttatggtgttatggaaacgggctctaacaatgacaggattcctgacaaggagaacacacctctcattgaacctcacgttcctcttcgtcctgctaacaccattaccaaggtcccttcagagaagatcctcagagctggaaaaattttacgaaatgccattctctctcgagcacctcacatgataagagatagaaaataccacctaaagacatacagacaatgctgtgtgggaactgaactggtggactggatgatgcagcagacaccatgtgttcactcccggactcaagctgttggcatgtggcaagtcctgttagaagatggtgttctcaaccacgtggaccaggagcaccatttccaagacaaatatttattctatcgatttctggatgatgagcacgaggatgcccctttgcctactgaggaggagaagaaggagtgtgatgaggagctccaggacaccatgctgctgctgtcacagatgggccccgacgcccacatgaggatgatccttcgcaaaccacctggccagaggactgtggatgacctagagattatctatgaggagcttcttcatattaaagccttatcccatctttctaccacagtgaaacgagagttagcaggtgttctcatttttgagtctcacgccaaaggagggactgtgttgtttaaccagggggaagaaggtacctcctggtacattattctaaaaggatcagtgaatgtagtcatttacggcaagggtgtggtctgcaccctgcatgaaggagatgacttcggcaagttagcactagtgaatgatgccccacgagctgcctctatcgtcttacgagaagataactgccatttcttaagagtagacaaggaggatttcaaccggatcctaagggacgtggaggcgaatacagtcagacttaaagaacatgaccaagatgtcttggtgctggagaaggtcccagcagggaacagagcttctaatcaaggaaactcacagcctcagcaaaagtatactgtgatgtcaggaacacctgaaaaaattttagagcattttctagaaacaatacgccttgaggcaactttaaatgaagcaacagattctgttttaaatgactttattatgatgcactgtgtttttatgccaaatacccagctttgcccggcactggtggcccactaccacgcacagccttcacaaggtacagaacaggagaaaatggattatgccctcaacaataagaggcgagtcatccgcctggttctacagtgggctgccatgtatggagacctcctgcaagaggatgacgtatctatggccttcctggaggagttttatgtatctgtatcagatgatgcccggatgattgctgccctcaaggagcaactgccagagttggagaagattgtcaagcaaatctcagaagatgcaaaggcaccacaaaagaagcacaaggttcttttgcaacagttcaatacgggcgatgagagagcccagaagcgccagcctatccgcggctctgatgaagttctgtttaaggtctattgcatggaccacacctacacaaccattcgggtgccagtggccacttcggtgaaggaagtcatcagtgcagttgccgacaagctgggctccggggagggcctgatcatagtcaagatgagttccggaggagaaaaggtggtgctcaaacctaatgatgtttcagtatttacgacgctcaccattaatggacgcctgtttgcttgcccgcgagagcaattcgattcactgactcccttaccagaacaggaaggcccaactgttggaacagtgggaacttttgaactgatgagctccaaagatttagcataccagatgacaatttatgattgggaactcttcaactgcgtgcatgagctggagctaatctatcacacatttggaaggcataattttaaaaagaccacagcaaacttggatttgttcctgaggagatttaatgaaattcagttttgggtcgtcactgagatctgcctttgttctcagctcagcaagcgtgttcagctattaaaaaaatttattaagatagcagcccactgtaaggagtataaaaatctgaattccttttttgccatcgtcatgggactaagtaacattgctgtgagccgcttggcactaacgtgggagaaactgccaagcaagttcaagaagttctatgcggagtttgaaagtttaatggacccttcaaggaaccacagggcctacaggctgacagtagctaagctggaacctcctctcatccccttcatgcctttgctcattaaagatatgacatttactcatgaggggaacaagacgttcattgacaatctagtaaactttgaaaaaatgcgcatgattgcaaatacggccagaacagtgagatactacaggagccaacccttcaatcctgatgcagctcaagctaataagaaccatcaggatgtccggagttatgtacggcaattaaatgtgattgacaaccagagaactttatcacagatgtcacacagattagagcctcgtcgaccatagacatttcaaatgcccaaagcaacagtttgtctccagtccacaatctttcaaaaatgccatttatgctactactgactgtattgccactagagaattctacaaaacaagcaaaaacacatcctgagacacctcagggctgcattcagcttaccagctacctagcaagagaaggaattatttgacatgcctaaagcttacacactctagcttaggaatccccagtttgtggctaccctgtttcatttccacttgtgccatcttctttgctgaagtgttgaataaggcccacgtgaagagtttggtagaaatagccttgtcaagagaactagcaagcccctgacatttttttctaagagtgtttatgggatgagatgtgctacaaatgggatagatttggataaaattttaactcaacatcttgatttggagcctggggtttcgggaaggtgttgaggaatctatagaagtccaaattataggttgtattcttccatcttcattgtcttacctctgaaggaatagaacctgaccacattattatgaaacattatggctgtttatgtaatttagggaggactggtctgtaatttctgaatgtatatagaataatatttatgtttacaatgtaacttttcaaaatttacaaatcaggattatatacataagaattccactaagaaatgccaagattcttaaatttgccagcgttaaagtagaaaataacatttcagaagagcacaatatgcataaaacatttttcaaaattgaaatattttcctgggcattaaaaacctttactattggctacaaatttattgtacctgatgaaaacatattttctggacttaaatgttattacaaatatcttaattttcagtaattgttttgcactttcaaagattgtaaatagttcattcaatcaatggtatagagttatttatttgctacataatagatactgtgccaaataattactttttatttattttatttagtacgtaattttgaagtacattttttcctgttttcacaattagactacatttaatgtgtaggaattgtatgtatgtatatcttctgtaaataacatctagtatcttcactaaatataattgtcgaccgggaaaaaaaaaa

SEQ ID NO: 4 - protein, 1011 aa, Homo sapiens
MVAAHAAHSSSSAEWIACLDKRPLERSSEDVDIIFTRLKEVKAFEKFHPNLLHQICLCGYYENLEKGITLFRQGDIGTNWYAVLAGSLDVKVSETSSHQDAVTICTLGIGTAFGESILDNTPRHATIVTRESSELLRIEQKDFKALWEKYRQYMAGLLAPPYGVMETGSNNDRIPDKENTPLIEPHVPLRPANTITKVPSEKILRAGKILRNAILSRAPHMIRDRKYHLKTYRQCCVGTELVDWMMQQTPCVHSRTQAVGMWQVLLEDGVLNHVDQEHHFQDKYLFYRFLDDEHEDAPLPTEEEKKECDEELQDTMLLLSQMGPDAHMRMILRKPPGQRTVDDLEIIYEELLHIKALSHLSTTVKRELAGVLIFESHAKGGTVLFNQGEEGTSWYIILKGSVNVVIYGKGVVCTLHEGDDFGKLALVNDAPRAASIVLREDNCHFLRVDKEDFNRILRDVEANTVRLKEHDQDVLVLEKVPAGNRASNQGNSQPQQKYTVMSGTPEKILEHFLETIRLEATLNEATDSVLNDFIMMHCVFMPNTQLCPALVAHYHAQPSQGTEQEKMDYALNNKRRVIRLVLQWAAMYGDLLQEDDVSMAFLEEFYVSVSDDARMIAALKEQLPELEKIVKQISEDAKAPQKKHKVLLQQFNTGDERAQKRQPIRGSDEVLFKVYCMDHTYTTIRVPVATSVKEVISAVADKLGSGEGLIIVKMSSGGEKVVLKPNDVSVFTTLTINGRLFACPREQFDSLTPLPEQEGPTVGTVGTFELMSSKDLAYQMTIYDWELFNCVHELELIYHTFGRHNFKKTTANLDLFLRRFNEIQFWVVTEICLCSQLSKRVQLLKKFIKIAAHCKEYKNLNSFFAIVMGLSNIAVSRLALTWEKLPSKFKKFYAEFESLMDPSRNHRAYRLTVAKLEPPLIPFMPLLIKDMTFTHEGNKTFIDNLVNFEKMRMIANTARTVRYYRSQPFNPDAAQANKNHQDVRSYVRQLNVIDNQRTLSQMSHRLEPRRP

SEQ ID NO: 5 - DNA, 5525 bp, Homo sapiens
  CDS (75)..(1406)
agttcccagtgaaaggacagggatggcaagatcttttagcatttaggggatgcctttgttagtaaccgttcacaatgggcagctcccggctgagggtctttgaccctcatttggagaggaaagattccgccgcggcgctctcagaccgagagctgcccttgcctaccttcgatgtgccttatttcaaatacatcgacgaggaggatgaggacgatgaatggagcagccgctcgcagtcttccaccgaggatgactcagtggactctctgctctctgacagatatgtggtggtgtccgggaccccggagaagattttggagcaccttttgaatgacttgcacctggaagaagtccaggacaaagaaacagagaccctcctggatgacttccttctcacgtacactgtcttcatgacaactgatgacttgtgccaggctctgttaaggcactattctgctaagaagtatcaaggcaaagaggaaaactcagatgttccgcgtaggaaacgtaaagtcttgcatcttgtttcccagtggattgctctgtacaaagactggttacctgaagatgaacattcaaaaatgtttttaaagaccatatataggaatgtactggatgatgtttatgaatatccaatacttgaaaaagaattgaaagaatttcaaaagatacttggaatgcaccgtcgtcacactgtagatgaatattcaccacaaaaaaagaataaagcccttttccaccaattcagtcttaaggagaactggctccagcatagaggaactgtgactgaaacggaggaaattttctgccacgtgtatataacagagcactcctatgtcagtgtgaaggcaaaagtttccagtatagcccaagagattctgctctgcagccagctgggcaagcgagtgcagctggtgaaaaaattcatcaaaattgcggctcactgcaaagcccagagaaacctgaattctttctttgccattgtgatgggtctcaacactgcttctgtcagtcgactgtcgcagacctgggagaaaatccctgggaagtttaagaaacttttctctgaacttgaaagtttaacagatccttccctaaatcacaaagcctacagagatgcattcaaaaagatgaagccaccaaaaatccctttcgtgcccttattgcttaaagatgtaacatttattcatgaaggaaataaaacttttttggataatcttgtcaattttgaaaagctgcatatgatcgcagacactgtccgaaccctgagacactgcaggactaaccagtttggtgacctgtctccaaaagagcatcaagagttaaagtcctatgttaatcacctgtatgtcattgacagccagcaggctctgtttgagctctcacacaggatcgagcctcgggtgtgagccccactgcctcacctcccctgtatctgcagcactttgagctacgggaatgtctatgccaagcacgttgctttcctgtgagaaaagaagttgctgagttttatcagtataacccaagacattcacaggaaagccagccaaagcgtgttcaggaagtgatgtcagccaccagagagggggagaggttttctccatgctactctcgggacaagaaggcagaaggagagtcagaagcattcttgagatggagaaggctggtttcttatgatcacattgttgatccagtccagttttcaatatgagatgtgccagcatcaagacaagacaacgtcttgacatgcaatgaccaaatatttcattaagagcgtgcatgaaacaggaaggagtttttactttgcctagttttagattactgtccataagctgtcaaagaagtcattcttttgaacacctgatgacagagacagcatctctagatctccagggaggagaggtttctgttgatacaacctgtgacatcaccaaaagccacttgtgtctagggagttagtgaggactgcagctagcatccatgctctgatgggcagatgaacaatgtcaaggtgtgcatcactttgcaccacaatcaactattgacacatgcttgcaggtgaaattagtttctgtacaactgatttgcagctataggcaaggtagatgaagttgctttgccagtaaggaaaaatagtaatctttaagaaattgactcattgtttaatttctggggattttctttatacttctaagcaggctcttatcttttattggacataatatgattttgaaaaagcacagtgcctgacacattgcaaacactcaccaactgcttgctgaggtgacagagtcacaaaagtctgcattcttgtgcctgatgatgcattttgcgtacctcatacaggctccttgcccacactatggaatgacagcagccagtgcagggaggttaagtgacatttaatgagtgaagcacttagcactctctaggtaataagatagtggtaattactagtgttttggcaaatgaaaaatgccctgaaatagccaaatgtctgattaatgttggcaacttagaagtcctataatccaactaccagccaaagcagggagcctttctataatttgcctttttttttttttttttcaaaatctgagtcttctaaaatcttattattcccatttttaccaattgaggctcctgtagcaaataagacctcttgatattttcaaggactggttagaggatttctttcaaccttcacatgaacaaaacagcctatgggtcaaaataatgaaatccacccctgcctgctagatacttgtcaccttgctaaaatgcaagggcctggtccattcattttccaaatgcaggagtcttggtgcacttctcactcttcctgcctgttcatctctttcatgcccacacagacctgtttcctttttgtctcatcaacgcctcattcatcctcattactgaggcgtgtccaatgctttttgacatctttatagcagtgctgtttcctgggctcaggaaccacactgagcttgagatactgctggaaggaaccatgtggagagaaggtttgggagaactttgagagagacttagtttggcccagcatgtaaaacttcagtcctgaacatttatagggttttatagaagggcatcctccagggctggtccattcagagaaatgctgcatgctgccgtcatggaatgtggcccacaggacaccagagccgtgagaaccggagagcagacttccctcacggctgggctgagcaaaccctccaaagccctcctcacgcagttactaacaatagcatgggcttacagcacaagcacgtgttctcacctttttcctatgccctggactaaggtttggccagtgtaatcatataaggccatcctgacattgtttctgtgtttcaaaatttggatttttatttacattagaactacattgctcctagtagaacattacctttaggggactaattttccatggagaactatttcagcatattgcatgctgctcagaccccaagtcagatatgcccaccaagccagatgaagctacacaaatgtggtatttaaatgcattttgtacagtgacttcagagtatctcacatgacatgggtgtaaactggctggggagaaaatgatgcttgttcacctcttcctccagccgtggttaggtggtcctaggggtagcagagggaagggaggattttgtgcagtcaagatttgcttttccatccttgtcttctgaatgtctaaaatctctgcatctttctgaagtttaacaactgtctccagaggtttgccaggcagcagctctcagaagtttccaaagctttgcagaatcttagatctggaattaaagaattcaagcccgaattgtgagaaccagatattactcaacagaaagctctttctaaggaatctgagctgttcactggtggacagtggtggggcttgagtgctccttgttaataggatgggccatgcaccctctctggatattcaccaaggcctcttcagaatagggtttgttctggctagaagcgtggtctagaagatggctaagctctttgccagctctcatttggagttttattattgcataaaatcttcgctcactctgcaaatcttacgtaatctggcaccttcggcaccaggtggtgcaggggcacttctaagtgggctctttttgttacagcacaactctcagacagtcctgtgggtctttggattcgtcagcattccagcaaactagccctgcttagaagttagcacaagacggcagaatgcaggaccccgtaggcaaaatcacaaccttgctattaaaaaaaatttttttttacatacacatttgcaggtgttccctagagtgtggtgttttgaatttgctctttgtcatctgtataattgccaaatgattatagtgatacacatgacctgcattcacttttttctagtttccttaattatgtttagaataaattcgtttccctagaccgagaaccacaaacaggtagtgtggagcatacaccgaatttagaagcatgtggataaggtcagtgctcacactgcctagtccacagggagaggatgctgcatgaatatatacttgcctctgagtggaggagaaatcgtggcatgaaagagagagtaccagtgatgacttcttatccctggagctgggctttcactgctacccatatcccagccctgcgagtctgttctagccagcacagacaccgcagatccggaactgaatgttcctaaatggcgcagccaatccaggcttttcagaaactgggcaaaaacattaaaatggggacgatcgggtcttccgcagtggtccaacacaggatttcttttaaatgtttcaaaaacatgtccttaaaatttcagcctgcttcttagcgagtgggccagttttgcttaaaactggtgggggggcgggggggaagtttttaaaaattgccaaaaagttagatgcaaatgtattactgtataaagcaaagctgtatatactaaacattttttagcagagtaatatttatttgcatagtctatttattgtattcgtattacactgttattaaatactgggatgaaatcagtgacctgaagcaagaaatcttgccttttaatgtatcattaattagggctgctgtgatattgtcagcttgcattaacaattagaagatagagaacccgccatcagggtgtctacctaacttctcagggactacacttggtagttttccaccatttaaagaactggtaaatatgaaacatttgttgagttaccagaattgccattaacagtgttttctttcccatattccatgctttctgcctctgtgtatatatataatatatatgtatatgactgtgctgtgtatttatcgaagctagtaagcaataatttatatgtaaaaatggccaagcaatataaggttaaaacttatataagtaacccttaccttatcttgtattttcaatttttttttaaaactgcttttccaaatatgagactatgttaaagacaaaaaaaaaaaaaaaa

SEQ ID NO: 6 - protein, 444 aa, Homo sapiens
MGSSRLRVFDPHLERKDSAAALSDRELPLPTFDVPYFKYIDEEDEDDEWSSRSQSSTEDDSVDSLLSDRYVVVSGTPEKILEHLLNDLHLEEVQDKETETLLDDFLLTYTVFMTTDDLCQALLRHYSAKKYQGKEENSDVPRRKRKVLHLVSQWIALYKDWLPEDEHSKMFLKTIYRNVLDDVYEYPILEKELKEFQKILGMHRRHTVDEYSPQKKNKALFHQFSLKENWLQHRGTVTETEEIFCHVYITEHSYVSVKAKVSSIAQEILLCSQLGKRVQLVKKFIKIAAHCKAQRNLNSFFAIVMGLNTASVSRLSQTWEKIPGKFKKLFSELESLTDPSLNHKAYRDAFKKMKPPKIPFVPLLLKDVTFIHEGNKTFLDNLVNFEKLHMIADTVRTLRHCRTNQFGDLSPKEHQELKSYVNHLYVIDSQQALFELSHRIEPRV

SEQ ID NO: 7 - DNA, 2019 bp, Homo sapiens
  CDS (1)..(1677)
gtgctggagagagcctctcagggcgccggcccttcccgacccccaaccccaggcaggaaccggtatacagtgatgtctggcaccccagagaagatcctagagcttctgttggaggccatgggaccagattccagtgctcatgacccaacagagacattcctcagcgacttcctcctgacccacagggtcttcatgcccagcgcccaactctgcgctgcccttctgcaccacttccatgtggagcctgcgggtggcagcgagcaggagcgcagcacctacgtctgcaacaagaggcagcagatcttgcggctggtcagccagtgggtggccctgtatggctccatgctccacactgaccctgtggccaccagcttcctccagaaactctcagacctggtgggcagggacacccgactcagcaacctgctgagggagcagtggccagagaggcggcgatgccacaggttggagaatggctgtgggaatgcatctcctcagatgaaggcccggaacttgcctgtttggctccccaaccaggacgagccccttcctggcagcagctgtgccatccaagttggggataaagtcccctatgacatctgccggccagaccactcagtgttgaccctgcagctgcctgtgacagcctccgtgagagaggtgatggcagcgttggcccaggaggatggctggaccaaggggcaggtgctggtgaaggtcaattctgcaggtgatgccattggcctgcagccagatgcccgtggtgtggccacatctctggggctcaatgagcgtctctttgttgtcaacccacaggaagtgcatgagctgatcccacaccctgaccagctggggcccactgtgggctctgctgaggggctggacctggtgagtgccaaggacctggcaggccagctgacggaccacgactggagcctcttcaacagtatccaccaggtggagctgatccactatgtgctgggcccccagcatctgcgggatgtcaccaccgccaacctggagcgcttcatgcgccgcttcaatgagctgcagtactgggtggccaccgagctgtgtctctgccccgtgcccggcccccgggcccagctgctcaggaagttcattaagctggcggcccacctcaaggagcagaagaatctcaattccttctttgccgtcatgtttggcctcagcaactcggccatcagccgcctagcccacacctgggagcggctgcctcacaaagtccggaagctgtactccgccctcgagaggctgctggatccctcatggaaccaccgggtataccgactggccctcgccaagctctcccctcctgtcatccccttcatgccccttcttctcaaagacatgaccttcattcatgagggaaaccacacactagtggagaatctcatcaactttgagaagatgagaatgatggccagagccgcgcggatgctgcaccactgccgaagccacaaccctgtgcctctctcaccactcagaagccgagtttcccacctccacgaggacagccaggtggcgaggatttccacatgctcggagcagtccctgagcacccggagtccagccagcacctgggcttatgtccagcagctgaaggtcattgacaaccagcgggaactctcccgcctctcccgagagctggagccatgaggaggggctgggactggagctggagcaggcacttgcagccgggaaagccagggtgtgccgggccaagatactcacaggctggccacagctgggcaaggctctccgtggagtggactcgagtccctggagcaggcagtgtggaggcagccatcccctgtgatgactggcagctaaggaggacctcggagtggacccgagccaggaataacgaatgacccaaggccaaggaagggaggacagagaggccccaggagtgggtggagagtggagtgcgctgggacgttgtgtgcaatagagaggtctccacaccagatgtcttccagaaaaaaaaaaaaaaaa

SEQ ID NO: 8 - protein, 559 aa, Homo sapiens
VLERASQGAGPSRPPTPGRNRYTVMSGTPEKILELLLEAMGPDSSAHDPTETFLSDFLLTHRVFMPSAQLCAALLHHFHVEPAGGSEQERSTYVCNKRQQILRLVSQWVALYGSMLHTDPVATSFLQKLSDLVGRDTRLSNLLREQWPERRRCHRLENGCGNASPQMKARNLPVWLPNQDEPLPGSSCAIQVGDKVPYDICRPDHSVLTLQLPVTASVREVMAALAQEDGWTKGQVLVKVNSAGDAIGLQPDARGVATSLGLNERLFVVNPQEVHELIPHPDQLGPTVGSAEGLDLVSAKDLAGQLTDHDWSLFNSIHQVELIHYVLGPQHLRDVTTANLERFMRRFNELQYWVATELCLCPVPGPRAQLLRKFIKLAAHLKEQKNLNSFFAVMFGLSNSAISRLAHTWERLPHKVRKLYSALERLLDPSWNHRVYRLALAKLSPPVIPFMPLLLKDMTFIHEGNHTLVENLINFEKMRMMARAARMLHHCRSHNPVPLSPLRSRVSHLHEDSQVARISTCSEQSLSTRSPASTWAYVQQLKVIDNQRELSRLSRELEP

SEQ ID NO: 9 - DNA, 33 bp, Artificial Sequence
  Description of Artificial Sequence Synthetic oligonucleotide
atggcttacccatacgatgttccagattacgcg

SEQ ID NO: 10 - DNA, 20 bp, Artificial Sequence
  Description of Artificial Sequence Synthetic primer
gctctttgaaccacacagca

SEQ ID NO: 11 - DNA, 20 bp, Artificial Sequence
  Description of Artificial Sequence Synthetic primer
tgtcttctcgcaggatgatg

SEQ ID NO: 12 - DNA, 21 bp, Artificial Sequence
  Description of Artificial Sequence Synthetic primer
gggctccttctccatcaccaa

SEQ ID NO: 13 - DNA, 21 bp, Artificial Sequence
  Description of Artificial Sequence Synthetic primer
cttcatcggtctgctcgctca

SEQ ID NO: 14 - DNA, 20 bp, Artificial Sequence
  Description of Artificial Sequence Synthetic primer
agcgaaagtgagaccagggc

SEQ ID NO: 15 - DNA, 20 bp, Artificial Sequence
  Description of Artificial Sequence Synthetic primer
ggcagggggagagatgagaa

SEQ ID NO: 16 - DNA, 21 bp, Artificial Sequence
  Description of Artificial Sequence Synthetic primer
gcacaacttcagcctcccaga

SEQ ID NO: 17 - DNA, 21 bp, Artificial Sequence
  Description of Artificial Sequence Synthetic primer
cttcccattcaccgctccatt

SEQ ID NO: 18 - DNA, 23 bp, Artificial Sequence
  Description of Artificial Sequence Synthetic oligonucleotide
aannnnnnnnnnnnnnnnnnnuu